SEQUENCE LISTING (39 sequences)

SEQ ID NO: 1 - protein, 37 aa, Homo sapiens [sequence omitted]

SEQ ID NO: 2 - protein, 37 aa, Mus musculus
KCNTATCATQRLANFLVRSSNNLGPVLPPTNVGSNTY

SEQ ID NO: 3 - protein, 11 aa, Homo sapiens
ATQRLANFLVH

SEQ ID NO: 4 - protein, 10 aa, Homo sapiens
SNNFGAILSS

SEQ ID NO: 5 - protein, 7 aa, Homo sapiens
NVGSNTY

SEQ ID NO: 6 - protein, 6 aa, Artificial
  Hexapeptide derived from human IAPP
ATQRLA

SEQ ID NO: 7 - protein, 6 aa, Artificial
  Hexapeptide derived from human IAPP
TQRLAN

SEQ ID NO: 8 - protein, 6 aa, Artificial
  Hexapeptide derived from human IAPP
QRLANF

SEQ ID NO: 9 - protein, 6 aa, Artificial
  Hexapeptide derived from human IAPP
RLANFL

SEQ ID NO: 10 - protein, 6 aa, Artificial
  Hexapeptide derived from human IAPP
LANFLV

SEQ ID NO: 11 - protein, 6 aa, Artificial
  Hexapeptide derived from human IAPP
ANFLVH

SEQ ID NO: 12 - protein, 6 aa, Artificial
  Hexapeptide derived from human IAPP
NFLVHS

SEQ ID NO: 13 - protein, 6 aa, Artificial
  Hexapeptide derived from human IAPP
FLVHSS

SEQ ID NO: 14 - protein, 6 aa, Artificial
  Hexapeptide derived from human IAPP
SSNNFG

SEQ ID NO: 15 - protein, 6 aa, Artificial
  Hexapeptide derived from human IAPP
SNNFGA

SEQ ID NO: 16 - protein, 6 aa, Artificial
  Hexapeptide derived from human IAPP
NNFGAI

SEQ ID NO: 17 - protein, 6 aa, Artificial
  Hexapeptide derived from human IAPP
NFGAIL

SEQ ID NO: 18 - protein, 6 aa, Artificial
  Hexapeptide derived from human IAPP
FGAILS

SEQ ID NO: 19 - protein, 6 aa, Artificial
  Hexapeptide derived from human IAPP
GAILSS

SEQ ID NO: 20 - protein, 6 aa, Artificial
  Hexapeptide derived from human IAPP
AILSST

SEQ ID NO: 21 - protein, 6 aa, Artificial
  Hexapeptide derived from human IAPP
ILSSTN

SEQ ID NO: 22 - protein, 5 aa, Artificial
  Pentapeptide derived from human IAPP
ANFLV

SEQ ID NO: 23 - protein, 4 aa, Artificial
  Tetrapeptide derived from human IAPP
ANFL

SEQ ID NO: 24 - protein, 3 aa, Artificial
  Tripeptide derived from human IAPP
ANF

SEQ ID NO: 25 - protein, 3 aa, Artificial
  Tripeptide derived from human IAPP
GNF

SEQ ID NO: 26 - protein, 3 aa, Artificial
  Tripeptide derived from human IAPP
AGF

SEQ ID NO: 27 - protein, 3 aa, Artificial
  Tripeptide derived from human IAPP
ANG

SEQ ID NO: 28 - protein, 3 aa, Artificial
  Tripeptide derived from human IAPP
  misc_feature (3)..(3): Xaa can be any naturally occurring amino acid
ANX

SEQ ID NO: 29 - protein, 3 aa, Artificial
  Tripeptide derived from human IAPP
  misc_feature (2)..(2): Xaa can be any naturally occurring amino acid
AXF

SEQ ID NO: 30 - protein, 3 aa, Artificial
  Tripeptide derived from human IAPP
  misc_feature (1)..(1): Xaa can be any naturally occurring amino acid
XNF

SEQ ID NO: 31 - protein, 5 aa, Artificial
  Pentapeptide derived from human IAPP
NFLVH

SEQ ID NO: 32 - protein, 4 aa, Artificial
  Tetrapeptide derived from human IAPP
FLVH

SEQ ID NO: 33 - protein, 3 aa, Artificial
  Tripeptide derived from human IAPP
NFL

SEQ ID NO: 34 - protein, 3 aa, Artificial
  Tripeptide derived from human IAPP
LVH

SEQ ID NO: 35 - protein, 3 aa, Artificial
  Tripeptide derived from human IAPP
FLV

SEQ ID NO: 36 - protein, 10 aa, Homo sapiens
GSNKGAIIGL

SEQ ID NO: 37 - protein, 10 aa, Homo sapiens
HVAAGAVVGG

SEQ ID NO: 38 - protein, 13 aa, Homo sapiens
ATQRLANFLVHSS

SEQ ID NO: 39 - protein, 13 aa, Homo sapiens
SSNNFGAILSSTN

(19) United States
(12) Patent Application Publication
(10) Pub. No.: US 2008/0004211 A1
(43) Pub. Date: Jan. 3, 2008

(54) Inhibitors of Amyloid Fibril Formation and Uses Thereof

(76) Inventor: Paul Fraser, Toronto (CA)

Correspondence Address: MCCARTHY TETRAULT LLP, BOX 48, SUITE 4700,, 66WELLINGTON STREET WEST, TORONTO, ON, M5K 1E6, CA

(21) Appl. No.: 10/590,435

(22) PCT Filed: Feb. 22, 2005
(86) PCT No.: PCT/CA05/00247

§ 371 Date: Aug. 23, 2006

Related U.S. Application Data
(60) Provisional application No. 60/546,186, filed on Feb. 23, 2004.

(51) Int. Cl. A61K 38/06; A61K 38/07; A61K 38/08; A61K 38/28; A61P 3/10; C07K 7/06; C12N 5/00; C12N 5/06; C12Q 1/02; C12Q 3/00; G01N 33/68

(52) U.S. Cl. 514/4; 435/29; 435/325; 435/375; 436/86; 514/17; 514/18; 530/330; 530/331

(57) ABSTRACT

The present invention provides new antifibrillogenic agents and peptides, compositions and cells containing same, compositions that bind to same, effective therapeutics for preventing or delaying the progression of, e.g., Alzheimer's disease and diabetes, methods for optimizing antifibrillogenic agents, and methods of using the antifibrillogenic agents, peptides, compositions, and cells of the invention for detecting and/or inhibiting amyloid fibril formation.

### FIELD OF THE INVENTION

The invention relates to new antifibrillogenic agents, a composition containing same, and a method of using these new antifibrillogenic agents. In one aspect, the agents can be used to inhibit amyloid fibril formation. In another aspect, they can be used as cytoprotectants. Screening methods, methods of identifying modulators of amyloid fibril formation, and peptide mimetics are also encompassed within the field of the invention.

### BACKGROUND OF THE INVENTION

Amyloidosis is a pathological condition characterized by the presence or deposition of amyloid fibers. “Amyloid” is a generic term referring to a group of diverse, but specific, protein deposits (intracellular and/or extracellular), which are seen in a number of different diseases. Though diverse in their occurrence, all amyloid deposits have common morphologic properties, stain with specific dyes (e.g., Congo red), and have a characteristic red-green birefringent appearance in polarized light after staining. They also share common ultrastructural features and common x-ray diffraction and infrared spectra.

Amyloid-related diseases can be restricted to one organ, or can spread to several organs. The first instance is referred to as “localized amyloidosis”, while the second is referred to as “systemic amyloidosis”.

Some amyloidotic diseases can be idiopathic, but most of these diseases appear as a complication of a previously-existing disorder. For example, primary amyloidosis can appear without any other pathology, or can follow plasma-cell dyscrasia or multiple myeloma. Secondary amyloidosis is usually associated with chronic infection (such as tuberculosis) or chronic inflammation (such as rheumatoid arthritis). A familial form of secondary amyloidosis is also seen in familial Mediterranean fever (FMF). This and other familial types of amyloidosis are genetically inherited, and are found in specific population groups. Furthermore, since deposits are found in several organs, they are also considered systemic amyloid diseases. Another type of systemic amyloidosis is found in long-term hemodialysis patients. In each of these cases, a different amyloidogenic protein is involved in amyloid deposition.

Different amyloids are also characterized by the type of protein present in the deposit. For example, neurodegenerative diseases, such as scrapie, bovine spongiform encephalitis (BSE), Creutzfeldt-Jakob disease, and the like, are characterized by the appearance and accumulation of a protease-resistant form of a prion protein (referred to as AScr or PrP-27) in the central nervous system. Similarly, Alzheimer's disease (AD), another neurodegenerative disorder, is characterized by neuritic plaques and neurofibrillary tangles. In this case, the plaque/blood-vessel amyloid is formed by the deposition of fibrillar amyloid-β protein. Other diseases, such as adult-onset diabetes (type 2 diabetes), are characterized by the localized accumulation of amyloid in the pancreas. Amyloid deposits are present in pancreatic islets of up to 96% of patients with non-insulin-dependent diabetes (NIDDM; i.e., type 2 diabetes) at post-mortem. These fibrillar accumulations result from the aggregation of the islet amyloid polypeptide (IAPP), also known as amylin.

There is no known, widely-accepted therapy or treatment which significantly dissolves amyloid deposits in situ. Nevertheless, the key role played by amyloid in both AD and type 2 diabetes suggests that prevention of plaque formation will have significant therapeutic benefits in these disease states.

Each amyloidogenic protein has the same ability to organize into β-sheets and to form insoluble fibrils that are deposited extracellularly or intracellularly. Furthermore, each amyloidogenic protein binds to other elements, including proteoglycan, amyloid P, and complement components. Although the amino acid sequence of each amyloidogenic protein is different, the sequences share similarities, including regions with the ability to bind to the glycosaminoglycan (GAG) portion of proteoglycan (referred to as the GAG binding site), and other regions which promote p-sheet formation. This suggests that amyloid fibrils are formed by a similar protein misfolding pathway, and that, therefore, therapeutic interventions to control their folding may be beneficial in all amyloid-related diseases.

In specific cases, amyloidotic fibrils, once deposited, may become toxic to the surrounding cells. For example, Aβ fibrils organized as senile plaques have been shown to be associated with dead neuronal cells and microgliosis in patients with Alzheimer's disease. When tested in vitro, Aβ peptide was shown to be capable of triggering an activation process of microglia (brain macrophages); this would explain the presence of microgliosis and brain inflammation in the brains of patients with Alzheimer's disease. There is also some suggestion that the deposits themselves are not toxic, but, rather, diffusible oligomers that can, for example, disrupt membrane integrity. In any case, the prevention of any form of aggregate may be beneficial in an in vivo setting.

In another type of amyloidosis seen in patients with type 2 diabetes, and in patients with type 1 diabetes post-transplantation, the amyloidogenic protein, IAPP, has been shown to induce β-islet cell toxicity in vitro. Hence, the appearance of IAPP fibrils in the pancreas of type 2 or type 1 diabetic patients has been associated with loss of the β-islet cells (Langerhans) and organ dysfunction.

Islet Amyloid Polypeptide and Diabetes

Islet hyalinosis (amyloid deposition) was first described over a century ago as the presence of fibrous-protein aggregates in the pancreas of patients with severe hyperglycemia (Opie, E. L., J. Exp. Med., 5:397-428, 1990). Today, islet amyloid, composed predominantly of IAPP (amylin), is a characteristic histopathological marker in over 90% of all cases of type 2 diabetes. The mature IAPP molecule is a 37-amino-acid peptide derived from a larger precursor peptide, pro-IAPP. IAPP co-localizes and is co-secreted with insulin in response to β-cell secretagogues. This pathological feature is not associated with insulin-dependent diabetes (type 1 diabetes), and is a unifying characteristic for the heterogeneous clinical phenotypes diagnosed as NIDDM (type 2 diabetes).

The causal factors for islet amyloidosis, and its role in the disease process, have yet to be determined. However, longitudinal studies in cats and immuno-cytochemical investigations in monkeys have shown that a progressive increase in islet amyloid is associated with a dramatic decrease in the population of insulin-secreting β-cells and with an increased severity of the disease. More recently, transgenic approaches have strengthened the relationship of IAPP plaque formation and β-cell dysfunction, which indicates that amyloid deposition is a principal factor in type 2 diabetes. Amyloid accumulations are also likely underestimated and much more extensive, since the low resolution histological dyes currently used are unable to detect anything other than large deposits.

IAPP is co-localized with insulin in β cell dense core secretory granules. Since IAPP is also co-secreted with insulin, it has been suggested that IAPP plays a role in regulating blood glucose by controlling insulin secretion. The presence of soluble IAPP in the plasma itself is normally not problematic. In patients with type 2 diabetes, however, the accumulation of pancreatic IAPP leads to a buildup of IAPP-amyloid as insoluble fibrous deposits; these deposits eventually replace the insulin-producing β cells of the islet, resulting in β cell depletion and failure (Westermark and Grimelius, Acta Path. Microbial Scand., sect A., 81:291-300, 1973; de Koning et al., Diabetologia, 36:378-84, 1993; and Lorenzo et al., Nature, 368:756-60, 1994). Amyloid lesions precede hyper-glycemia, suggesting that IAPP deposition is a principal cause of islet dysfunction (de Koning et al., Diabetologia, 36:378-84, 1993). Further, accumulation of IAPP results in a significantly decreased β cell mass in both human and non-human primates (Clark et al., Diabetes Res., 9:151-59, 1988). Cumulatively, these observations suggest a close link between islet amyloid and the progression of type 2 diabetes.

Genetic and biochemical investigations have implicated amyloid as a primary and causative agent in numerous disease states (e.g., Alzheimer's disease). However, it has become apparent that the relationship between IAPP and diabetes is part of a more complex cascade involving several interconnected factors. Research to date has indicated that type 2 diabetes is initiated by other factors, such as peripheral insulin resistance or obesity. These factors result in a heightened metabolism of β cells and a subsequent increase in insulin secretion. This chain of events is predicted to result in an abnormally high and localized concentration of the co-secreted IAPP that culminates in extracellular and vascular amyloid deposition. These fibrillar accumulations, either through direct toxicity and/or by impeding the diffusion of nutrient, contribute to islet dysfunction and, ultimately, to the cellular pathology of type 2 diabetes.

It has been suggested that differing levels of glycosylation may lead to a pool of peptides that are more apt to be involved in aggregation. Studies have also suggested that, in type 2 diabetes, incomplete enzymatic processing of IAPP from its precursor pro-IAPP, by the prohormone convertase PC2, may provide a level of aggregatable peptide needed for the “seeding” of amyloid fibrils. Still other studies have examined the properties contained in the amino acid sequence of human IAPP that make the peptide prone to aggregation when compared with rodent IAPP, which does not form typical amyloid fibrils (Johnson et al., N. Engl. J. Med., 321:513-18, 1989; and Moriarty and Raleigh, Biochemistry, 38:1811-818, 1999).

IAPP amyloid has many features in common with cerebral amyloid, which is formed in Alzheimer's disease from the amyloid-β (Aβ) peptide. Disease states involving both types of amyloid are progressive, age-related, and associated with irreversible deterioration in cellular function. Neither pathological condition requires synthesis of a mutated form of the peptide, and both component peptides are derived from a larger precursor and form morphologically-similar amyloid fibrils.

Islet Amyloid Polypeptide and Cell Death

Diseases caused by the death or malfunctioning of a particular type of cells can be treated by transplanting into the patient healthy cells of the relevant type. This approach has been used for type I diabetes, and also for insulin-dependent type 2 diabetics patients. These cells are cultured in vitro prior to transplantation, to allow them to recover after the isolation procedure, or to reduce their immunogenicity. However, in many instances, the transplants are unsuccessful, due to the death of the transplanted cells. One reason for this poor success rate may be IAPP, which can form fibrils and become toxic to the cells in vitro. In addition, IAPP fibrils are likely to continue to grow after the cells are transplanted, and cause death or dysfunction of the cells. This may occur even when the cells are from a healthy donor, and when the patient receiving the transplant does not have a disease that is characterized by the presence of fibrils.

Islet Amyloid Polypeptide

The mature IAPP molecule is a 37-amino-acid peptide synthesized in the pancreas. (Human IAPP is SEQ. ID. NO. 1.) IAPP contains three principal domains that contribute to fibril formation (FIG. 1; SEQ. ID. NOs. 3, 4, and 5). These domains have been identified by looking at different peptide fragments and the effects of proline mutations in the rodent IAPP sequence (SEQ. ID. NO. 2), which does not form amyloid fibrils. The initial N-terminal-domain disulfide bridge (residues 2 and 7) is not critical to amyloid fibril formation.

The inventor previously demonstrated that small fragments of the IAPP displayed an inhibitory activity when combined with full-length IAPP 1-37 (Fraser, WO 02/24727). It was shown that these fragments were capable of interacting with IAPP and preventing aggregation by disrupting the peptide-peptide packing within the extending amyloid fibril. This effectively ‘caps-off’ the polymerization necessary for amyloid assembly. Other approaches have been used to inhibit IAPP fibril formation (Kapurniotu et al., U.S. Pat. No. 6,359,112, 1998), described peptides SNNFGAILSS (hIAPP, 20-29; SEQ. ID. NO. 4), GSNKGAIIGL (β-IAPP, 25-34; SEQ. ID. NO. 36), and HVAAGAVVGG (PrP, 110-119) (SEQ. ID. NO. 37) for inhibiting and analyzing amyloid formation. Kapurniotu et al. further described peptides of, generally, between 3-15 amino acids, and containing at least the active peptide sequence GA. Cooper et al. (European Patent Application No.0 289 287) disclose various hepta- and hexa-peptides of IAPP, including ANFLVH and NFLVHS, for the use in diagnosing diabetes mellitus.

However, there exists a need for minimal inhibitory domains which would allow for small molecule mimetics of amyloid polypeptides. Smaller molecules make it more feasible to use a combinatorial approach to the optimization of activity. Specifically, smaller peptides fall within the molecular weight range of small organic compounds, so mimetics can be synthesized that have better in vivo properties Additionally, a minimal inhibitory domain is desirable because smaller molecules are easier to deal with in terms of bioavailability, and may be more likely to avoid the attenuation of metabolism—a common failing of peptide-based approaches. Accordingly, there exists a need for the identification of small peptides that can modulate amyloid polypeptide activity, and that can be used in treatment, screening, and drug development for IAPP-associated conditions and amyloid-related disorders.

### SUMMARY OF THE INVENTION

The invention relates to, inter alia, in vitro and in vivo inhibitors of amyloid fibril formation. These inhibitors are, e.g., antifibrillogenic agents and peptides which are capable of controlling IAPP aggregation and amyloid formation. This property may be used advantageously in other embodiments of the invention, as disclosed herein.

In one embodiment, the invention provides antifibrillogenic agents and peptides that are truncated (penta-, tetra-, or tri -) peptides of the hexapeptides disclosed in WO 02/24727, particularly, ANFLVH (SEQ. ID. NO. 11), NFLVHS (SEQ. ID. NO. 12), SNNFGA (SEQ. ID. NO. 15), and GAILSS (SEQ. ID. NO. 19), as well as isomers, retro or retro-inverso isomers, peptidomimetics, or salts thereof. In one embodiment, the antifibrillogenic agents or peptides are cytoprotectants. In a further embodiment, the antifibrillogenic agents or peptides inhibit amyloidosis.

In another embodiment, the antifibrillogenic agents or peptides are truncated peptides of the hexapeptide ANFLVH (SEQ. ID. NO. 11), or isomers, retro or retro-inverso isomers, peptidomimetics, or salts thereof. In a further embodiment, the peptides are ANFLV (SEQ. ID. NO. 22), ANFL (SEQ. ID. NO. 23), ANF (SEQ. ID. NO. 24), or NFL (SEQ. ID. NO. 33). In still another embodiment, the antifibrillogenic agents or peptides are ANFLV (SEQ. ID. NO. 22), ANF (SEQ. ID. NO. 24), or NFL (SEQ. ID. NO. 33). In yet another embodiment, the antifibrillogenic agents or peptides are ANFLV (SEQ. ID. NO. 22) or ANF (SEQ. ID. NO. 24).

The antifibrillogenic agents or peptides may also be all-[D] isomers, all-[L] isomers, or a mixture of [L] and [D] isomers of the peptide.

In one embodiment, the antifibrillogenic agent or peptide of the invention is a tripeptide having the formula selected from the group consisting of:

(I) ANX (SEQ. ID. NO. 28),

(II) AXF (SEQ. ID. NO. 29), and

(III) XNF (SEQ. ID. NO. 30),

where X is any amino acid. In one embodiment, X is any amino acid except for cysteine. In another embodiment, X is glycine (G).

In a further embodiment, the antifibrillogenic agent or peptide is a tripeptide selected from the group consisting of ANF (SEQ. ID. NO. 24), GNF (SEQ. ID. NO. 25), AGF (SEQ. ID. NO. 26), ANG (SEQ. ID. NO. 27), an isomer thereof, a retro or a retro-inverso isomer thereof, a peptidomimetic thereof, and a salt thereof. In another embodiment, the tripeptide is selected from the group consisting of ANF (SEQ. ID. NO. 24), GNF (SEQ. ID. NO. 25), AGF (SEQ. ID. NO. 26), an isomer thereof, a retro or a retro-inverso isomer thereof, a peptidomimetic thereof, and a salt thereof.

The antifibrillogenic agents and peptides of the invention may advantageously be used in the treatment of, e.g., cultured pancreatic islet cells in vitro prior to transplantation, for the treatment of type I and type 2 diabetes patients (e.g., post-transplantation), and for the prevention or inhibition of fibril formation in the transplanted cells. Additionally, the antifibrillogenic agents and peptides of the invention may advantageously be used for cytoprotection and/or for inhibiting amyloidosis, including IAPP-related amyloidosis.

The antifibrillogenic agents, peptides, and methods of the invention may also advantageously be used in preventing or delaying the progression of, notably, type 1 and type 2 diabetes in transplanted cases, and in inhibiting fibril formation for controlling folding or deposition of amyloid proteins.

The invention also provides compounds for inhibiting amyloidosis and/or for cytoprotection, where the compounds bind to the sequence ANFLVH (SEQ. ID. NO. 11), or to a truncated (penta-, tetra-, or tri-) peptide thereof. Upon binding to the sequence, fibril formation and amyloidosis are prevented. The compounds (e.g., enzymes, antibodies, etc.) may desirably bind to ANF (SEQ. ID. NO 24), ANX (SEQ. ID. NO. 28), AXF (SEQ. ID. NO. 29), or XNF (SEQ. ID. NO. 30), where X is any amino acid. In one embodiment, X is glycine.

The invention also relates to labeled conjugates for in vivo imaging of amyloid deposits featuring a conjugate of formula I:
Aₜ [symbol] Aₗₙₖ [symbol] _{z}A_{lab} (I)
where z is 0 or 1; Aₜ is an antifibrillogenic agent as defined above; Aₗₙₖ is a linker moiety; and A_{lab} is a labeling moiety that allows for said in vivo imaging. Desirably, A_{lab} is a radiolabeling moiety, and is, more preferably, ^{99m}Tc, ⁹⁹Tc, ⁶⁴Cu, ⁶⁷Cu, ⁹⁷Ru, ¹¹⁹Pd, ¹⁸⁶Re, ¹⁸⁸Re, ¹¹¹In, ^{113m}In, ¹⁵³Gd, ⁹⁰Y, ¹⁵³Sm, ¹⁶⁶Ho, ¹⁹⁸Au, ⁹⁰Sr, ⁸⁹Sr, ¹¹⁵Rh, ²⁰¹Tl, ⁵¹Cr, ⁶⁷Ga, ⁵⁷Co, ⁶⁰Co, ¹²³I, ¹²⁵I, ¹³¹I, or ¹⁸F. The labeled conjugate may also be formulated in a composition for in vivo imaging of amyloid deposits. Such a composition may comprise a therapeutically-effective amount of a labeled conjugate, as defined above, in association with a pharmaceutically-acceptable carrier.

The invention also includes compositions for the treatment of amyloidosis disorders in a patient, including a therapeutically-effective amount of an antifibrillogenic agent or peptide of the invention, as defined above, with a pharmaceutically-acceptable carrier. Also provided are methods for the treatment of an amyloidosis disorder in a patient, wherein a therapeutically-effective amount of the antifibrillogenic agent is administered to a patient in need of such treatment. In one embodiment, the compositions of the invention may be administered in conjunction with insulin, or in conjunction with sulfonylurea and/or glucose sensitizers (e.g., in a treatment for diabetes).

Processes for the preparation of cells suitable for transplantation into a mammal, which cells are capable of forming amyloid deposits or of evoking endogenous amyloid deposition once transplanted, are also disclosed herein. The processes include contacting such cells in vitro with an antifibrillogenic agent or peptide of the invention. The antifibrillogenic agent causes a breakdown of amyloid deposits (the deposits having been formed by the cells prior to coming into contact with the antifibrillogenic agent). In order to optimize the survival of cells, the cells may desirably be cultured in the presence of the antifibrillogenic agent. This may also be useful for producing a more homogeneous preparation of B cells from which exocrine and other cells have been removed. Also provided are cells prepared in accordance with these processes.

The invention further includes methods for treating type 1 and insulin-dependent type 2 diabetes patients post-transplantation, wherein an antifibrillogenic agent or peptide of the invention is administered to a type 1 or type 2 diabetes patient, so that amyloid deposit formation and amyloidosis is inhibited, prevented, and/or reduced.

The use of antifibrillogenic agents and peptides, compositions containing same, or compounds as described above for the various methods described herein, or for manufacturing a medicament or a composition for use in the various methods described herein, are also disclosed.

The invention also includes methods for determining an optimized peptide for inhibition of amyloidosis, including amyloidogenesis, by systematic substitution of each residue of an original tripeptide of the invention. An optimized tripeptide is one having inhibition greater than that of the original tripeptide. Such tripeptides can be chosen from the group consisting of ANF (SEQ. ID. NO. 24), GNF (SEQ. ID. NO. 25), AGF (SEQ. ID. NO. 26), and NFL (SEQ. ID. NO. 33). Optimized tripeptides are also encompassed within the scope of the invention. Systematic substitution of a tripeptide will result in 57 different derivatives that can be tested for inhibition and compared to inhibition of the original tripeptide. The invention also includes the optimized peptides determined by this method.

Other features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 is a comparison of the human (SEQ. ID. NO. 1) and mouse (SEQ. ID. NO. 2) IAPP sequences. The figure illustrates the primary and amyloid-forming domains of human IAPP, and the β-sheet-breaking proline substitutions of the mouse peptide.

FIG. 2 shows the primary sequence of human IAPP (SEQ. ID. NO. 1) and the original series of peptides examined as described in Fraser (WO 02/24727) (SEQ. ID. NOs. 6-21).

FIG. 3 shows optimization of IAPP peptide inhibitors. Depicted are the peptide fragments that were examined to determine minimal IAPP binding and amyloid inhibition.

FIGS. 4A-B are graphs illustrating circular dichroism (CD) data for the truncated tripeptide, ANF (FRA2612) (SEQ. ID. NO. 24), indicating the transition from random coil to β-sheet. The ANF inhibited this conformational change when combined with IAPP at a molar ratio of 1:10.

FIGS. 4C-H show that modifications of the initial ANF (SEQ. ID. NO. 24) inhibitory sequence—to generate the peptides GNF (FRA2613) (SEQ. ID. NO. 25) (4C and 4D), AGF (FRA2614) (SEQ. ID. NO. 26) (4E and 4F), and NFL (SEQ. ID. NO. 33) (4G and 4H)—resulted in similar, but more potent, activities at significantly lower molar ratios.

FIGS. 5A-F are electron micrographs from negatively-stained preparations of the full-length IAPP showing the dense network of amyloid fibrils. The inhibitory peptide, ANF (SEQ. ID. NO. 24), reduced the relative levels of fibrils and altered the morphology of the aggregates which were formed. Data for substituted tripeptides GNF, AGF, and ANG (SEQ. ID. NOs. 25-27) are also shown. (A) IAPP control, (B) IAPP+ANF (SEQ. ID. NO. 24), (C) IAPP+GNF (SEQ. ID. NO. 25), (D) IAPP+AGF (SEQ. ID. NO. 26), (E) IAPP+ANG (SEQ. ID. NO. 27), and (F) IAPP+NFL (SEQ. ID. NO. 33) [molar ratio 1:10].

FIG. 6 is a graph illustrating the results of the toxicity assay for ANF and related peptides, ANFLVH (SEQ. ID. NO. 11), ANFLV (SEQ. ID. NO. 22), and ANFL (SEQ. ID. NO. 23); rat insulinoma (RIN) cells were exposed to exogenous IAPP, with and without peptide inhibitors, at a molar ratio of 1:20 [IAPP:inhibitor].

FIG. 7 is a graph illustrating the results of the toxicity assay for ANF and related peptides, ANFLVH (SEQ. ID. NO. 11), GNF (SEQ. ID. NO. 25), AGF (SEQ. ID. NO. 26), and ANG (SEQ. ID. NO. 27); rat insulinoma (RIN) cells were exposed to exogenous IAPP, with and without peptide inhibitors, at a molar ratio of 1:20 [IAPP:inhibitor].

FIG. 8 is a schematic diagram of the human IAPP construct used to generate human IAPP transgenic mice (pNASSMAR.RIP.hIAPP). In the figure, “insulin promoter” refers to the rat insulin promoter (RIP).

FIG. 9 is an ELISA illustrating IAPP expression in wild-type (WT) and transgenic (Tg+) mice under high-fat and normal-diet conditions.

FIG. 10 is a bar graph illustrating the amount of IAPP secreted by cultured islets that were isolated from IAPP transgenic (IAPP TG) and non-transgenic (nonTG) mice on control or high-fat diets in the presence (+) and absence (−) of glucose.

FIG. 11 illustrates the results of a cell-viability assay in IAPP transgenic and non-transgenic mice at high- and low-glucose concentrations.

FIGS. 12A-B are electron microscope images of amyloid fibrils in cultured human islet cells.

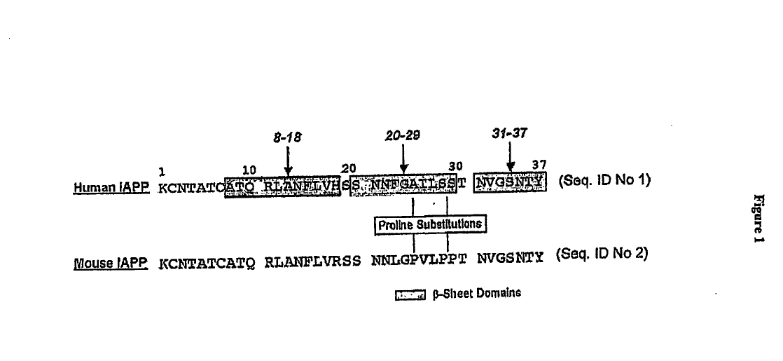

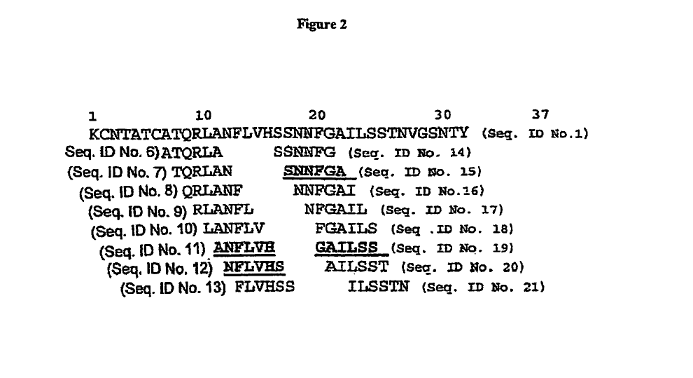

### DETAILED DESCRIPTION OF THE INVENTION

For the purpose of the present disclosure, the following terms are defined:

The term “peptidomimetic” includes non-peptide compounds which mimic the structural or the functional properties of a peptide.

The term “amyloid-related disorders” includes diseases associated with the accumulation of amyloid, whether the amyloid is restricted to one organ (“localized amyloidosis”) or is present in several organs (“systemic amyloidosis”). As further used herein, the term “amyloidosis” includes “amyloidogenesis” (the production of amyloid), the deposition of amyloid fibers, and any conditions characterized by the production, presence, and/or deposition of amyloid fibers.

Secondary amyloidosis may be associated with chronic infection (such as tuberculosis) or chronic inflammation (such as rheumatoid arthritis), including a familial form of secondary amyloidosis which is also seen in familial Mediterranean fever (FMF) and another type of systemic amyloidosis found in long-term hemodialysis patients. Localized forms of amyloidosis include, without limitation, diabetes type 2, and any related disorders thereof; neurodegenerative diseases, such as scrapie, bovine spongiform encephalitis (BSE), Creutzfeldt-Jakob disease, Alzheimer's disease, cerebral amyloid angiopathy, and prion-protein-related disorders. This term also includes systemic reactive (AA) amyloidoses, primary (AL) amyloidoses, hereditary systemic amyloidoses, senile systemic amyloidosis, cerebral amyloidosis, dialysis-related amyloidosis, and hormone-derived amyloidoses.

“Retro isomer” includes molecules (e.g., peptides) having a reversal of the direction of the molecular (e.g., peptide) backbone.

“Peptide” includes isomers thereof; retro or retro-inverso isomers thereof; peptidomimetics thereof; all-[D] isomers thereof; all-[L] isomers thereof; a mixture of [L] and [D] isomers thereof; and salts thereof.

“Inverso isomer” includes molecules (e.g., peptides) having an inversion of the amino acid chirality used to make the peptide.

“Retro-inverso isomer” includes molecules (e.g., peptides) having a reversal of both the peptide-backbone direction and the amino-acid chirality.

“Antifibrillogenic activity” includes the ability to block or prevent an amyloidogenic protein from forming fibrils, protofibrils, or oligomers, preferably by preventing it from adopting its β-pleated conformation, by disrupting protofilament interactions, and/or by interfering with the side-chain interactions within the folded peptide, which are believed to be necessary for aggregation and fibril formation.

The term “cytoprotection” or “cytoprotective activity” includes molecules (e.g., peptides) having the ability to protect cells from amyloid-induced toxicity.

The terms “antifibrillogenic agent” and “inhibitor of fibril formation” are used herein interchangeably.

The present invention provides new antifibrillogenic agents and inhibitors of fibril formation, for controlling folding or deposition of amyloid proteins. The present invention also provides methods to prevent or delay the progression of diabetes and other amyloidosis disorders. The present invention further provides small peptides having inhibitory properties, and agents capable of controlling IAPP aggregation and amyloid formation.

Antifibrillogenic agents of the invention are provided for inhibiting amyloidosis and/or for cytoprotection. Such antifibrillogenic agents include peptides made from truncating such hexapeptides as ANFLVH (SEQ. ID. NO. 11), NFLVHS (SEQ. ID. NO. 12), SNNFGA (SEQ. ID. NO. 15), and GAILSS (SEQ. ID. NO. 19). In one embodiment, the antifibrillogenic agents are peptides made from truncating the hexapeptides ANFLVH (SEQ. ID. NO. 11) and NFLVHS (SEQ. ID. NO. 12). In one embodiment, hexapeptides are truncated to ANFLV (SEQ. ID. NO. 22), ANFL, or tripeptides such as ANF (SEQ. ID. NO. 24), NFL (SEQ. ID. NO. 33), FLV (SEQ. ID. NO. 35), and LVH (SEQ. ID. NO. 34). In another embodiment, the peptides are ANFLV (SEQ. ID. NO. 22), ANF (SEQ. ID. NO. 24), or NFL (SEQ. ID. NO. 33).

In accordance with the present invention, peptides for inhibiting amyloidosis and/or for cytoprotection are also provided. Each such peptide has a sequence selected from truncated hexapeptide ANFLVH (SEQ. ID. NO. 1), as described above.

Another embodiment of the invention relates to peptides for inhibiting amyloidosis and/or for cytoprotection, where the peptide binds to a sequence selected from ANFLVH (SEQ. ID. NO. 11), NFLVHS (SEQ. ID. NO. 12), SNNFGA (SEQ. ID. NO. 15), and GAILSS (SEQ. ID. NO. 19), and, upon binding, prevents fibril formation and amyloidosis.

In a further embodiment of the invention, the invention relates to methods of determining optimized tripeptides for inhibiting amyloidosis and/or for cytoprotection, comprising the steps of:
(a) choosing an original peptide from the group consisting of ANF (SEQ. ID. NO. 24), NFL (SEQ. ID. NO. 33), FLV (SEQ. ID. NO. 35), and LVH (SEQ. ID. NO. 34);
(b) systematically substituting at each residue a different amino acid;
(c) testing the ability of each derivative to inhibit amyloid fibril formation; and
(d) comparing the inhibition of each derivative with the inhibition of the original peptide,
   wherein an increase in inhibition of the derivative over the original peptide indicates an optimized peptide.

For example, if the original peptide consisted of the amino acid sequence 123, test derivatives would be X23, 1X3, and 12X, wherein X is selected from the group consisting of naturally-occurring amino acids Gly, Ala, Val, Leu, Ile, Ser, Thr, Met, Asp, Asn, Glu, Gin, Arg, Lys, His, Phe, Tyr, Trp, and Pro, as well as other amino acids that do not occur naturally. In one embodiment, the original peptide is ANF (SEQ. ID. NO. 24) and the optimized peptides are either XNF(SEQ. ID. NO. 30), AXF (SEQ. ID. NO. 29), or ANX (SEQ. ID. NO. 28). This systematic substitution generates 57 different derivatives for each original tripeptide. Inhibitory activity can be determined using at least one of the in vitro assay systems described below, including, without limitation, CD, EM, and cell toxicity. In a further embodiment, the invention relates to the optimized peptide determined by this method.

The antifibrillogenic agents can be formulated in a composition for inhibiting amyloidosis and/or for cytoprotection. Such a composition would include a therapeutically-effective amount of antifibrillogenic agents of the invention in association with a pharmaceutically-acceptable carrier.

Another embodiment of the invention relates to compounds for inhibiting amyloidosis and/or for cytoprotection, wherein the compounds bind with a peptide as defined above. The compounds may be, e.g., an enzyme that binds to or controls the expression of the peptide, or an antibody that binds to the peptide. Such an antibody may be specific for the peptide, and may be either a monoclonal or polyclonal antibody.

Agents of the invention may be used for the ex vivo preparation (e.g., preparation in culture) of cells suitable for transplantation into a mammal (e.g., islet cells), which cells are capable of forming amyloid deposits. In accordance with the invention, the cells are contacted with the antifibrillogenic agent, in preparation for transplantation. The antifibrillogenic agent causes a breakdown of amyloid deposits—the deposits having been formed by the cells prior to coming in contact with the antifibrillogenic agent.

The agents of the invention may advantageously be used in a method for treating type 1 and type 2 diabetes patients post-transplantation. In accordance with this method, an antifibrillogenic agent is administered to a type 1 or type 2 diabetic patient for inhibiting, preventing, and/or reducing amyloid deposit formation and amyloidosis. The antifibrillogenic agent may be administered in conjunction with insulin.

The antifibrillogenic agents of the present invention may also be used in a method for inhibiting amyloidosis and/or for cytoprotection. In accordance with this method, a therapeutically-effective amount of the antifibrillogenic agent is administered to a subject, such that the antifibrillogenic agent prevents or reduces amyloid deposition. The antifibrillogenic agent may desirably be administered by cell therapy or gene therapy, wherein the cells have been modified to produce and secrete the antifibrillogenic agent. Such cells may be modified ex vivo or in vivo.

The antifibrillogenic agents of the invention may also be used for imaging plaques, in which case the antifibrillogenic agents (e.g., peptides) are amyloid-targeting imaging agents of the following formula:
Aₜ [symbol] Aₗₙₖ [symbol] _{z}A_{lab} (I)
where z is 0 or 1; Aₜ is the antifibrillogenic agent of IAPP fibril formation, as described herein; Aₗₙₖ is a linker moiety; and A_{lab} is a labeling moiety. Labeling moiety A_{lab} allows the amyloid-targeting imaging agent, once at the target site in vivo, to be visualized by instrumentation, such as CT, MRI, ultrasound, or radioisotopic or fluorescence detection. The labeling moiety either modulates an externally-applied energy, or generates a detectable energy itself. The labeling moiety may be an echogenic substance in the case of an ultrasound-contrast agent, a paramagnetic metal chelate in the case of an MRI-contrast agent, a radioactive atom (e.g., radioactive fluorine), or a chelated radioactive metal ion (e.g., In-Ill) in the case of a radionuclide imaging agent, a radio-opaque chelate or compound (e.g., a polyiodinated aromatic) for an x-ray contrast agent, or a fluorescent or colored dye in the case of an optical imaging contrast agent. In one embodiment, labeling moiety A_{lab} may be a metal chelator. In an advantageous embodiment, A_{lab} is a radionuclide (either a chelate of a metal ion or a single atom) or a paramagnetic metal ion chelate. According to one aspect of the invention, a labeled targeting-molecule/chelator conjugate comprises a labeling moiety A_{lab} (e.g., a radionuclide) attached directly to amyloid-targeting moiety Aₜ, thereby not requiring the use of a linker moiety.

Preferably, A_{lab} includes a radionuclide selected from ^{99m}Tc, ⁹⁹Tc, ⁶⁴Cu, ⁶⁷Cu, ⁹⁷Ru, ¹¹⁹Pd, ¹⁸⁶Re, ¹⁸⁸Re, ¹¹¹In, ^{113m}In, ¹⁵³Gd, ⁹⁰Y, ¹⁵³Sm, ¹⁶⁶Ho, ¹⁹⁸Au, ⁹⁰Sr, ⁸⁹Sr, ¹¹⁵Rh, ²⁰¹Tl, ⁵¹Cr, ⁶⁷Ga, ⁵⁷Co, ⁶⁰Co, ¹²³I, ¹²⁵I, ¹³¹I, and ¹⁸F.

As an imaging agent, A_{lab} preferably includes a radionuclide selected from the group consisting of Tc and Re. More preferably, A_{lab} is a metal chelate of a radioactive or paramagnetic metal ion.

In both AD and type 2 diabetes, amyloid plays a key role. The antifibrillogenic agents of the invention may be peptides, peptidomimetics, antibodies, or other compounds that interact or interfere with either or both regions of the amyloidogenic peptide that are involved in amyloid formation—e.g., ATQRLANFLVHSS (SEQ. ID. NO. 38) and SSNNFGAILSSTN (SEQ. ID. NO. 39) in the case of the IAPP peptide. The antifibrillogenic agents may also be enzymes that bind to or control the expression of the amyloidogenic peptide.

When the antifibrillogenic agents are peptides, all-[D] peptides, all-[L] peptides, and peptides which are a mixture of [L] and [D] isomers are included. Without wishing to be bound by a particular theory or interpretation of how the invention operates, antifibrillogenic agents are believed to “interfere” with the amyloidogenic peptide by binding and disrupting its folding into the amyloidogenic β-sheet conformation, disrupting protofilament interactions, and/or impeding side-chain interactions within the folded peptide that are necessary for aggregation and fibril formation.

The antifibrillogenic agents of the invention may be peptides, which can be modified or substituted analogs. Some analogs include unnatural amino acids or modifications of N- or C-terminal amino acids. Unnatural amino acids include D-amino acids, α,α-disubstituted amino acids, N-alkyl amino acids, lactic acid, 4-hydroxyproline, gamma-carboxyglutamate, ε-N,N,N-trimethyllysine, ε-N-acetyllysine, O-phosphoserine, N-acetylserine, N-formylmethionine, 3-methylhistidine, 5-hydroxylysine, ψ-N-methylarginine, and isoaspartic acid.

As applied to polypeptides, the term “substantial identity” means that two peptide sequences, when optimally aligned, as by the programs GAP or BESTFIT, using default gap weights, share at least 80% sequence identity, preferably at least 90 percent sequence identity, and more preferably at least 95 percent sequence identity or more (e.g., 99% sequence identity). Preferably, residue positions which are not identical differ by conservative amino acid substitutions. The phrase “conservative amino acid substitutions” refers to the interchangeability of residues having similar side chains. For example, a group of amino acids having aliphatic side chains includes glycine, alanine, valine, leucine, and isoleucine; a group of amino acids having aliphatic-hydroxyl side chains includes serine and threonine; a group of amino acids having amide-containing side chains includes asparagine and glutamine; a group of amino acids having aromatic side chains includes phenylalanine, tyrosine, and tryptophan; a group of amino acids having basic side chains includes lysine, arginine, and histidine; and a group of amino acids having sulfur-containing side chains includes cysteine and methionine. Preferred conservative amino acids substitution groups include valine-leucine-isoleucine, phenylalanine-tyrosine, lysine-arginine, alanine-valine, and asparagine-glutamine.

The term “antibody” or “immunoglobulin”, as used herein, includes intact antibodies and binding fragments thereof. Typically, fragments compete with the intact antibody from which they were derived for specific binding to an antigen. Exemplary binding fragments include, without limitation, separate heavy chains, light chains Fab, Fab′, F(ab′)₂, Fabc, Fc, and Fv. Fragments are produced by recombinant DNA techniques, or by enzymatic or chemical separation of intact immunoglobulins. The term “antibody” also includes one or more immuno-globulin chains that are chemically conjugated to, or expressed as, fusion proteins with other proteins. The term “antibody” also includes a bispecific antibody. A bispecific or bifunctional antibody is an artificial hybrid antibody having two different heavy-/light-chain pairs and two different binding sites. Bispecific antibodies can be produced by a variety of methods, including fusion of hybridomas or linking of Fab′ fragments. See, e.g., Songsivilai and Lachmann, Clin. Exp. Immunol., 79:315-21, 1990; Kostelny et al., J. Immunol., 148:1547-553, 1992. Specific binding between two entities refers to an affinity of at least 10⁶, 10⁷, 10⁸, 10⁹M⁻¹, or 10¹⁰M⁻¹. Affinities greater than 10⁸ M⁻¹ are preferred.

The phrase “pharmaceutical composition”, as used herein, refers to a chemical or biological composition suitable for administration to a mammalian individual. Such compositions may be specifically formulated for administration via one or more of a number of routes, including, but not limited to, oral, parenteral, intravenous, intraarterial, subcutaneous, intranasal, sublingual, intraspinal, intracerebroventricular, and the like. A “pharmaceutical excipient” or a “pharmaceutically-acceptable excipient” is a carrier, usually a liquid, in which an active therapeutic peptide is formulated. The excipient generally does not provide any pharmacological activity to the formulation, although it may provide chemical and/or biological stability, release characteristics, and the like. Exemplary formulations can be found, for example, in Remington's Pharmaceutical Sciences, 19^{th} ed., Grennaro, A., ed., 1995.

The peptides, proteins, fragments, analogs, and other amyloidogenic peptides of the invention may be synthesized by solid-phase peptide synthesis or recombinant expression, according to standard methods well known in the art; they may also be obtained from natural sources. Automatic peptide synthesizers may be used, and are commercially available from numerous manufacturers, such as Applied Biosystems (Perkin Elmer; Foster City, Calif.); procedures for preparing synthetic peptides are also known in the art.

Antifibrillogenic agents of the invention may also be derived from the peptides by substitution of one or more residues in the naturally-occurring sequence. In one embodiment, the agents are peptidomimetics of the peptides. The agents may be modified by removing or inserting one or more amino acid residues, or by substituting one or more amino acid residues with other amino acids or non-amino-acid fragments, such as thienylalanine, cyclohexylalanine, and phenylglycine.

The antifibrillogenic agents (e.g., peptides) may be used actively to immunize patients (e.g., as vaccines), so that the patients, after immunization, will produce antibodies that will recognize the peptide sequences against which the antibodies have been raised. Alternatively, peptide antifibrillogenic agents of the invention can be used for producing antibodies to be administered to patients for passive immunization. The antibodies administered (in the case of a passive immunization) or the antibodies produced by the patients (in the case of an active immunization) will recognize a sequence on IAPP corresponding to the sequence against which they have been raised, for inhibiting or reducing plaque formation.

Exemplary Amyloidoses

As non-limiting illustrations of the utility of the invention, the following types of amyloidosis are described in more detail below.

AA (Systemic Reactive) Amyloidosis

Generally, AA amyloidosis is a manifestation of a number of diseases that provoke a sustained acute-phase response. Such diseases include chronic inflammatory disorders, chronic local or systemic microbial infections, and malignant neoplasms.

AA fibrils are generally composed of 8000-dalton fragments (AA peptide or protein) formed by proteolytic cleavage of serum amyloid A protein (apoSSA), a circulating apolipoprotein which is present in high-density lipoprotein (HDL) complexes and which is synthesized in hepatocytes in response to such cytokines as IL-1, IL-6, and TNF. Deposition can be widespread in the body, with a preference for parenchymal organs. The spleen is usually a deposition site, and the kidneys may also be affected. Deposition is also common in the heart and gastrointestinal tract.

AA amyloid diseases include, but are not limited to, inflammatory diseases, such as rheumatoid arthritis, juvenile chronic arthritis, ankylosing spondylitis, psoriasis, psoriatic arthropathy, Reiter's syndrome, adult Still's disease, Behcet's syndrome, and Crohn's disease. AA deposits are also produced as a result of chronic microbial infections, such as leprosy, tuberculosis, bronchiectasis, decubitus ulcers, chronic pyelonephritis, osteomyelitis, and Whipple's disease. Certain malignant neoplasms can also result in AA fibril amyloid deposits. These include such conditions as Hodgkin's lymphoma; renal carcinoma; carcinomas of gut, lung, and urogenital tract; basal cell carcinoma; and hairy cell leukemia.

AL (Primary) Amyloidosis

AL amyloid deposition is generally associated with almost any dyscrasia of the B lymphocyte lineage, ranging from malignancy of plasma cells (multiple myeloma) to benign monoclonal gammopathy. At times, the presence of amyloid deposits may be a primary indicator of the underlying dyscrasia.

Fibrils of AL amyloid deposits are composed of monoclonal immunoglobulin light chains or fragments thereof. More specifically, the fragments are derived from the N-terminal region of the light chain (kappa or lambda), and contain all or part of the variable (VL) domain thereof. Deposits generally occur in the mesenchymal tissues, causing peripheral and autonomic neuropathy, carpal tunnel syndrome, macroglossia, restrictive cardiomyopathy, arthropathy of large joints, immune dyscrasias, myelomas, and occult dyscrasias. However, it should be noted that almost any tissue, particularly the tissue of visceral organs such as the heart, may be involved.

Hereditary Systemic Amyloidoses

There are many forms of hereditary systemic amyloidoses, although they are relatively rare conditions. Adult onset of symptoms and their inheritance patterns (usually autosomal dominant) lead to persistence of such disorders in the general population. Generally, the syndromes are attributable to point mutations in the precursor protein leading to production of variant amyloidogenic peptides or proteins. Table 1 summarizes the fibril composition of exemplary forms of these disorders.

TABLE 1

| Fibril Peptide/Protein | Genetic variant | Clinical Syndrome |
|---|---|---|
| Transthyretin and fragments | Met30, many others | Familial amyloid polyneuropathy |
| (ATTR) | | (FAP), (Mainly peripheral nerves) |
| Transthyretin and fragments | Thr45, Ala60, Ser84, | Cardiac involvement predominant |
| (ATTR) | Met111, Ile122 | without neuropathy |
| N-terminal fragment of | Arg26 | Familial amyloid polyneuropathy (FAP), |
| Apolipoprotein A1 (apoA1) | | (mainly peripheral nerves) |
| N-terminal fragment of | Arg26, Arg50, Arg60, | Ostertag-type, non-neuropathic |
| Apoliproprotein A1 (AapoA1) | others | (predominantly visceral involvement) |
| Lysozyme (Alys) | Thr56, His67 | Ostertag-type, non-neuropathic |
| | | (predominantly visceral involvement) |
| Fibrogen ∀ chain fragment | Leu554, Val526 | Cranial neuropathy with lattice |
| | | corneal dystrophy |
| Gelsolin fragment (Agel) | Asn187, Tyr187 | Cranial neuropathy with lattice |
| | | corneal dystrophy |
| Cystatin C fragment | Glu68 | Hereditary cerebral hemorrhage |
| | | (cerebral amyloid angiopathy)- |
| | | Icelandic type |
| β-amyloid protein (aβ) | Gln693 | Hereditary cerebral hemorrhage |
| derived from Amyloid | | (cerebral amyloid angiopathy)-Dutch |
| Precursor Protein (APP) | | type |
| β-amyloid protein (aβ) | Ile717, Phe717, Gly717 | Familial Alzheimer's Disease |
| derived from Amyloid | | |
| Precursor Protein (APP) | | |
| β-amyloid protein (aβ) | Asn670, Leu671 | Familial Dementia - probably |
| derived from Amyloid | | Alzheimer's Disease |
| Precursor Protein (APP) | | |
| Prion Protein (PrP) derived | Leu102, Val167, Asn178, | Familial Creutzfeldt-Jakob disease; |
| from Prp Precursor protein | Lys200 | Gerstmann-Sträussler-Scheinker |
| 51-91 insert | | syndrome (hereditary spongiform |
| | | encephalopathies, prion diseases) |
| AA derived from Serum | | Familial Mediterranean fever, |
| amyloid A protein (ApoSSA) | | predominant renal involvement |
| | | (autosomal recessive) |
| AA derived from Serum | | Muckle-Wells syndrome, |
| amyloid A protein (ApoSSA) | | nephropathy, deafness, urticaria, limb |
| | | pain |
| Unknown | | Cardiomyopathy with persistent atrial |
| | | standstill |
| Unknown | | Cutaneous deposits (bullous, papular, |
| | | pustulodermal) |

*Data derived from Tan and Pepys, Histopathology, 25(5): 403-14, 1994.

The data provided in Table 1 are exemplary, and are not intended to limit the scope of the invention. For example, more than 40 separate point mutations in the transthyretin gene have been described, all of which give rise to clinically similar forms of familial amyloid polyneuropathy.

Transthyretin (TTR) is a 14-kilodalton protein that is also sometimes referred to as prealbumin. It is produced by the liver and choroid plexus, and functions in transporting thyroid hormones and vitamin A. At least 50 variant forms of the protein, each characterized by a single amino acid change, are responsible for various forms of familial amyloid polyneuropathy. For example, substitution of proline for leucine at position 55 results in a particularly progressive form of neuropathy; substitution of methionine for leucine at position 111 results in a severe cardiopathy in Danish patients. Amyloid deposits isolated from heart tissue of patients with systemic amyloidosis were composed of a heterogeneous mixture of TTR and fragments thereof, collectively referred to as ATTR, the full-length sequences of which have been characterized. ATTR fibril components can be extracted from such plaques, and their structure and sequence determined according to the methods known in the art.

Persons having point mutations in the molecule apolipoprotein A1 (e.g., Gly→Arg26; Trp4→Arg50; Leu→4 Arg60) exhibit a form of amyloidosis (“Östertag type”) characterized by deposits of the protein apolipoprotein A1 or fragments thereof (AapoA1). These patients have low levels of high-density lipoprotein (HDL), and present with a peripheral neuropathy or renal failure.

A mutation in the alpha chain of the enzyme lysozyme (e.g., Ile→Thr56 or Asp→His57) is the basis of another form of Östertag-type non-neuropathic hereditary amyloid reported in English families. Here, fibrils of the mutant lysozyme protein (Alys) are deposited. This protein, unlike most of the fibril-forming proteins described herein, is usually present in whole (unfragmented) form. Patients generally exhibit impaired renal function.

β-amyloid peptide (Aβ) is a 39- to 43-amino-acid peptide derived by proteolysis from a large protein known as beta amyloid precursor protein (βAPP). Mutations in βAPP result in familial forms of Alzheimer's disease, Down's syndrome, and/or senile dementia; these diseases are characterized by cerebral deposition of plaques composed of Aβ fibrils and other components, which are described in further detail below. Known mutations in APP associated with Alzheimer's disease occur proximate to the cleavage sites of β or gamma-secretase, or within Aβ. For example, position 717 is proximate to the site of gamma-secretase cleavage of APP in its processing to Aβ, and positions 670/671 are proximate to the site of β-secretase cleavage. Mutations at any of these residues may result in Alzheimer's disease, presumably by causing an increase in the amount of the 42-/43-amino-acid form of Aβ generated from APP. The structure and sequence of Aβ peptides of various lengths are well known in the art. Such peptides can be made according to methods known in the art (e.g., Glenner and Wong, Biochem. Biophys. Res. Comm., 129:885-90,1984; Glenner and Wong, Biochem. Biophys. Res. Comm., 122:1131-135,1984). In addition, various forms of the peptides are commercially available.

Synuclein is a synapse-associated protein that resembles an apolipoprotein, and is abundant in neuronal cytosol and presynaptic terminals. A peptide fragment derived from alpha-synuclein, termed NAC, is also a component of amyloid plaques of Alzheimer's disease.

Gelsolin is a calcium-binding protein that binds to actin filaments and fragments. Mutations at position 187 (e.g., Asp→Asn; Asp→Tyr) of the protein result in a form of hereditary systemic amyloidosis usually found in patients from Finland, and in persons of Dutch or Japanese origin. In afflicted individuals, fibrils formed from gelsolin fragments (Agel) usually consist of amino acids 173-243 (68-kDa carboxyterminal fragment), and are deposited in blood vessels and basement membranes, resulting in corneal dystrophy and cranial neuropathy which progresses to peripheral neuropathy, dystrophic skin changes, and deposition in other organs.

Other mutated proteins, such as a mutant alpha chain of fibrinogen (AfibA) and mutant cystatin C (Acys), also form fibrils and produce characteristic hereditary disorders. AfibA fibrils form deposits that are characteristic of a non-neuropathic hereditary amyloid with renal disease; Acys deposits are characteristic of an hereditary cerebral amyloid angiopathy reported in Iceland. In at least some cases, patients with cerebral amyloid angiopathy (CAA) have been shown to have amyloid fibrils containing a non-mutant form of cystatin C in conjunction with beta protein.

Certain forms of prion disease are now considered to be inheritable, accounting for up to 15% of the cases that were previously thought to be predominantly infectious in nature (Baldwin et al., in Research Advances in Alzheimer's Disease and Related Disorders, John Wiley and Sons, New York, 1995). In such prion disorders, patients develop plaques composed of abnormal isoforms of the normal prion protein (prPSc). A predominant mutant isoform, PrPSc, also referred to as AScr, differs from the normal cellular protein in its resistance to protease degradation, insolubility after detergent extraction, deposition in secondary lysosomes, post-translational synthesis, and high β-pleated sheet content. Genetic linkage has been established for at least five mutations resulting in Creutzfeldt-JaKob disease (CJD), Gerstmann-Sträussler-Scheinker syndrome (GSS), and fatal familial insomnia (FFI) (Baldwin, supra). Methods for extracting fibril peptides from scrapie fibrils, determining sequences, and making such peptides are known in the art. For example, one form of GSS has been linked to a PrP mutation at codon 102, while telencephalic GSS segregates with a mutation at codon 117. Mutations at codons 198 and 217 result in a form of GSS in which neuritic plaques characteristic of Alzheimer's disease contain PrP instead of Aβ peptide. Certain forms of familial CJD have been associated with mutations at codons 200 and 210; mutations at codons 129 and 178 have been found in both familial CJD and FFI (Baldwin, supra).

Senile Systemic Amyloidosis

Amyloid deposition, either systemic or focal, increases with age. For example, fibrils of wild-type transthyretin (TTR) are commonly found in heart tissue of elderly individuals. These may be asymptomatic or clinically silent, and may result in heart failure. Asymptomatic fibrillar focal deposits may also occur in the brain (Aβ), corpora amylacea of the prostate (Aβ2 microglobulin), joints, and seminal vesicles.

Cerebral Amyloidosis

Local deposition of amyloid is common in the brain, particularly in elderly individuals. The most frequent type of amyloid in the brain is composed primarily of Aβ peptide fibrils, resulting in dementia or sporadic (non-hereditary) Alzheimer's disease. In fact, the incidence of sporadic Alzheimer's disease greatly exceeds forms shown to be hereditary. Fibril peptides forming these plaques are very similar to those described above, with reference to hereditary forms of Alzheimer's disease (AD).

Dialysis-Related Amyloidosis

Plaques composed of β2 micro globulin (Aβ2M) fibrils commonly develop in patients receiving long-term hemodialysis or peritoneal dialysis. β2 microglobulin is a 11.8-kilodalton polypeptide; it is also the light-chain of Class I MHC antigens, which are present on all nucleated cells. Under normal circumstances, it is continuously shed from cell membranes and is normally filtered by the kidney. Failure of clearance, as in the case of impaired renal function, leads to deposition in the kidney and other sites (primarily in collagen-rich tissues of the joints). Unlike other fibril proteins, Aβ2M molecules are generally present in unfragmented form in the fibrils.

Hormone-Derived Amyloidoses

Endocrine organs may harbour amyloid deposits, particularly in aged individuals. Hormone-secreting tumours may also contain hormone-derived amyloid plaques, the fibrils of which are made up of polypeptide hormones such as calcitonin (medullary carcinoma of the thyroid), islet amyloid polypeptide (occurring in most patients with type 2 diabetes), and atrial natriuretic peptide (isolated atrial amyloidosis). Sequences and structures of these proteins are well known in the art.

Miscellaneous Amyloidoses

There are a variety of other forms of amyloid disease that are normally manifest as localized deposits of amyloid. In general, these diseases are probably the result of the localized production and/or lack of catabolism of specific fibril precursors or a predisposition of a particular tissue (such as the joint) for fibril deposition. Examples of such idiopathic deposition include nodular AL amyloid, cutaneous amyloid, endocrine amyloid, and tumour-related amyloid.

The invention, in a particular embodiment, is especially useful for treatment of diabetes (e.g., amyloid-related diabetes). The following description sets forth this aspect of the invention in more detail.

Type 2 Diabetes and IAPP

Primary Structure of IAPP and Fibril Formation

There are three regions of human IAPP (hIAPP) that have the potential to form fibrils. In addition to the region 20-29, originally described as the amyloidogenic region (Betsholtz et al., FEBS Lett., 251:261-64, 1989) and the report of hIAPP 30-37 forming fibrils (Nilsson and Raleigh, J. Mol. Biol., 294:1375-385, 1999), hIAPP 8-20 also forms fibrils (Fraser, WO 02/24727). These findings suggest that 20-29 is not the only amyloidogenic region of the sequence. In addition, the fragment rat IAPP 8-20, which has an arginine at position 18 but is otherwise homologous to hIAPP 8-20, formed fibrils in aqueous media.

Some studies have shown that fragments of hIAPP form fibrils rapidly in aqueous media; however, Fraser, supra, utilized a preparation of hIAPP free of ‘seeds’ (Higham et al., Eur. J. Biochem., 267:4998-5004, 2000), rather than a preparation of undefined solubility, as the starting material. Under these conditions, all peptide fragments were initially in random conformation, when examined with CD, and had no fibrillar structures present when examined by EM. This permitted examination of the effects of pH and counter ions on the change in peptide conformation from an unfolded state to the oligomerization and formation of fibrils. Previous studies have used HFIP to stabilize IAPP in artificial helical conformation, or have used seeds to generate conformational changes (Kayed et al., J. Mol. Biol., 287:81-796, 1999), which may not reflect the situation in vivo. The use of preformed seeds could preclude the formation of initial aggregation stages important in the in vivo generation of amyloid.

The three adjacent domains of hIAPP that have amyloidogenic potential may have a role in intermolecular binding, oligomerization, and fibril formation, and in interacting to form intramolecular β-sheets. Fraser, supra, was the first report of fragments of rat IAPP (rat IAPP 8-20) forming fibrils. As the 30-37 region of rat IAPP is identical in amino acid structure to hIAPP—and, therefore, capable of fibril formation—it could be predicted that these two β-strands interact and that rat IAPP should form fibrils. The lack of fibril formation from rat IAPP suggests that the proline substitutions at rat IAPP 25, 28, and 29 prevent β-strand formation in this region of the peptide; these proline substitutions not only inhibit intermolecular β-sheet formation and fibrils, but also disrupt intramolecular structure that would lead to fibril formation.

The histidine residue at position 13 in Aβ is important for fibril assembly. Mutant forms of Aβ without histidine residues do not form structures larger than protofilaments. In rodents, His13 of Aβ is replaced with an arginine residue, in a manner similar to the Arg18His substitution that occurs in IAPP. This substitution is believed to contribute to the lack of Aβ amyloid in rodents.

Fibril formation of hIAPP 1-37 is independent of pH, although the morphology differs. Fraser, supra, demonstrated that counter-ions present in the buffer influenced the morphology as well as the rate of fibril formation. Human IAPP 1-37 formed fibrils at similar rates in water and in 11 mM sodium-acetate, and on a shorter time scale in 2 mM Tris buffer. This was accompanied by a conversion from random to β-sheet conformation, as determined by CD analysis. Human IAPP 1-37 rapidly precipitated from 2 mM borate, citrate, and phosphate buffers with a loss of CD signal. As the acetate and citrate buffers, and the Tris and phosphate buffers, were similar in ionic strength and matched for pH, the differences in effect were attributed to the charge or shape of the buffer ions. Citrate and phosphate are more densely charged than acetate and Tris, respectively.

Binding of zinc to the histidine residue in the Aβ peptide has been proposed as an important factor for fibril assembly. The presence of His 18 was shown not to be essential, as rat IAPP 8-20 also formed fibrils. However, in the presence of zinc, fragments 18-29 and 20-29 formed longer, more loosely-packed fibrils, suggesting that zinc is able to affect the packing of peptide fragments into protofilaments, and the assembly of protofilaments into fibrils, independently of any interaction it may have with His18 (Fraser, supra). The highly-charged zinc ion could interact with hydrophobic residues, preventing lateral aggregation. A high concentration of zinc is present in the β-cell secretory granule, which could influence the folding of IAPP.

Secondary Structure Propensities of hIAPP

Previous studies examining secondary structure predictions have produced various potential conformations for hIAPP (Hubbard et al., Biochem. J., 275:785-88, 1991; Saldanha and Mahadevan, Protein Eng., 4:539-44, 1991). Structure predictions indicate that an alpha helix should be present at the N terminus of hIAPP. However, the CD data in the Fraser reference, supra, indicate that hIAPP is usually found either in a random coil state or in a β-sheet, or is precipitated from solution (Higham et al., Febs Lett., 470:55-60, 2000). Only in the presence of helix-promoting solvents (TFE, HFIP) does it exhibit alpha helical nature (Higham et al., Febs Lett., 470:55-60, 2000). This suggests either that hIAPP, in vitro, does not retain its native structure, or that hIAPP is unstructured and, under appropriate conditions, assumes a β-sheet structure more easily than other conformations. Alternatively, hIAPP in vivo could exist as a random coil structure, and circulate bound to a carrier to maintain stability. Although the secondary structures predicted by algorithms are based on known structures, they cannot predict whether a molecular conformation is kinetically accessible, and, therefore, possible to attain in vitro or in vivo.

The conformations determined separately for different domains of the peptide may not represent that existing in the intact molecule, since fragmentation removes tertiary contacts and fibril formation of separate fragments may occur under conditions where the full-length sequence does not form fibrils. Rat IAPP 8-20 will form fibrils, but the full-length rat IAPP does not. Despite the limitations of both secondary-structure predictions, and the difficulties of inferring structure from fragments, these methods can be used to model peptides.

Proposal of a Model for hIAPP Fibril Formation

The presence of two/three β-strands in the hIAPP sequence suggests that a small β-sheet is at the core of the monomeric structure. This could be stabilized by side-chain hydrogen bonding between the uncharged polar side chains of asparagine and/or glutamine residues.

Fibril formation of two/three β-strands is independent of pH and counter ions, and is driven by hydrophobic interactions. In the hIAPP sequence, 11 of 37 residues are hydrophobic. Increased hydrophobicity during the initial stages of hIAPP fibril formation has been demonstrated (Kayed et al., J. Mol. Biol., 287:781-96, 1999), suggesting that protofilament and fibril assembly exposes hydrophobic groups. Uncharged polar residues, such as glutamine, serine, asparagine, and threonine, participate in side-chain hydrogen bonding. Griffiths et al. (Journal of the American Chemical Society, 12:3539-354, 1995) suggested that residues 24-27 form a highly-ordered antiparallel β-sheet structure when examined as a 20-29 fragment.

An amyloidogenic domain of hIAPP has been identified using a series of overlapping peptide fragments, providing insight into molecular sequences important in amyloid fibril formation (Fraser, supra). Although the hIAPP 20-29 domain is clearly important, it is unlikely to act in isolation; other IAPP regions must contribute to formation/stabilization of the β-sheet conformation and the accompanying aggregation and fibril formation.

Fraser, supra, shows that there are at least two regions of IAPP involved in fibril formation, one β-pleated sheet region (IAPP 20-29) and one region of previously unknown function (IAPP 8-20). The antifibrillogenic agents of the present invention can act by interacting or interfering with either or both regions.

The fibril-forming ability of hIAPP was found to be pH insensitive, suggesting that the transition of IAPP in vivo, from the β-cell secretory granule (pH 5.5) to the extracellular space (pH 7.4), does not have a significant effect on the conformation of the peptide. It is more likely that changes in the granule components or in the extracellular environment, which are unique to type 2 diabetes, allow fibril formation to occur. The β-cell granule contains more than 30 identified proteins, and has high concentrations of both zinc and calcium. Intracellular molecular crowding could be essential for maintenance of hIAPP in its native conformation or for inhibition of aggregation. Changes which promote ‘seeding’ of amyloidogenic fragments or conformational rearrangements of intact hIAPP 1-37 initiate the progressive deposition of secreted IAPP as amyloid deposits, and the destruction of insulin-secreting cells. Similarly, in the early stages of type 2 diabetes, crowding effects in the extracellular space due to hypersecretion from the β-cells could result in increased concentration of hIAPP and aggregation leading to fibril formation.

The ultimate goal in the present invention is to control the disease process in order to prevent, delay, or reverse the progression of Alzheimer's disease, diabetes, or other amyloidosis disorders. Non-limiting examples of amyloidosis disorders are cerebral angiopathy, secondary amyloidosis, familial Mediterranean fever, Muckle-Wells syndrome, primary amyloidosis, familial amyloid polyneuropathy, hereditary cerebral hemorrhage, chronic hemodialysis-associated amyloidosis, and prion disorders such as Creutzfeldt-Jacob disease and Gerstmann-Sträussler-Scheinker syndrome.

In accordance with the invention, a series of IAPP-derived peptide fragments has been identified. These fragments have the ability to bind to the full-length protein and prevent normal folding and amyloid fibril formation. The activity of these inhibitors has been assessed, as detailed herein, using a series of biophysical techniques that include protein spectroscopy, fluorescence assays, and electron microscopy.

Previous investigations demonstrated that small fragments of the amyloid-β peptide displayed an inhibitory activity when combined with the full-length Aβ 1-42 or 1-40. It has been postulated that these fragments were capable of interacting with Aβ and preventing aggregation by disrupting the peptide-peptide packing within the extending amyloid fibril. This would effectively ‘cap-off’ the polymerization necessary for amyloid assembly.

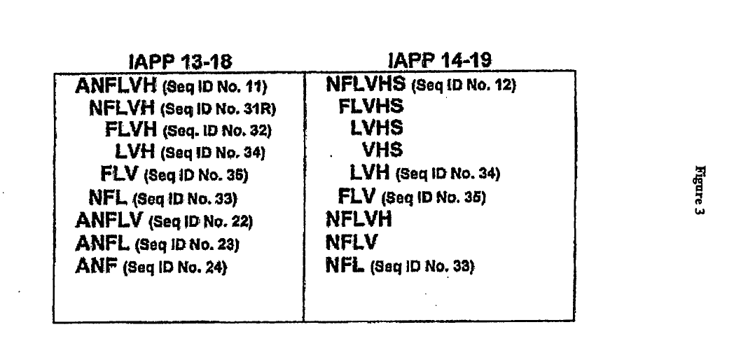

To determine if a similar strategy could be employed with IAPP, two series of overlapping hexapeptides, derived from key β-sheet structural domains within, were previously synthesized and investigated (see FIG. 2). The IAPP 20-29 domain has been extensively studied, and is considered to be a critical region for fibril formation. This has been supported, for example, by the β-sheet-breaking proline residues in rodent IAPP, which prevent amyloid formation. More recently, investigations have indicated a second β-sheet domain spanning residues 8-20. A third β-sheet-forming sequence (residues 31-37) has been identified, but this region was less amenable to fragment analysis due to virtually irreversible aggregation. Soluble peptides capable of binding to IAPP, and disrupting amyloid packing and fibril formation, may be generated by targeting the two domains contained within residues 8-29.

Using in vitro assay systems, the activities of the peptide fragments were assessed and four (4) potent inhibitors were identified (Fraser, supra). Two peptides from the IAPP20-29 region displayed inhibitory activity—SNNFGA and GAILSS; two peptides from the 8-20 domain displayed inhibitory activity—ANFLVH and NFLVHS. When combined with full-length IAPP1-37 at relatively low molar ratios of 1:5 and 1:1 [inhibitor:IAPP], these peptides were able to: (1) prevent the folding of human IAPP into a β-sheet conformation; (2) virtually eliminate the assembly of IAPP fibrils, as determined by electron microscopy; and (3) significantly attenuate the toxicity of IAPP fibrils in cell culture. Slight changes in sequence could result in an amyloid-enhancing effect, where the peptide fragments could independently assemble into amyloid fibrils. These investigations have generated a number of interesting molecules which can be optimized in terms of their inhibitory properties.

Optimization of the Inhibitory Peptide Fragments

The inventor's initial investigations identified four hexapeptide fragments of IAPP which were able to inhibit amyloidogenesis and cell toxicity effectively. To advance this technology, the following has been examined: (1) the minimal inhibitory sequence for each peptide; (2) the residues which confer activity within these sequences; (3) optimized activity through a process of systematic residue substitution; and (4) modelling of the molecular structure of the most active peptides in an effort ultimately to generate small molecule analogues.

The truncated peptides of the invention and/or optimized peptides of the invention can be formulated into pharmaceutical compositions with pharmaceutically-acceptable carriers known in the art. They can be used in effective amounts (amounts to achieve the desired result) to modulate (e.g., inhibit) amyloid fibril formation. In another embodiment, the peptides can be used to treat amyloid-related disorders, such as diabetes or Alzheimer's. Alternatively, the peptides can be used in screening assays to identify suitable IAPP fibril formation modulators, diagnostics, and peptide mimetics to develop or design molecules that can be used in the treatment of amyloid-related disorders.

The following non-limiting examples are illustrative of the present invention:

### EXAMPLES

Methods: Assays for Evaluating Inhibitor Activity

1. Circular Dichroism (CD): Amyloidogenic peptides and proteins undergo a conformational transition from a native to β-sheet conformation. This misfolding promotes protein-protein aggregation and the formation of amyloid fibrils that have a similar morphology and structure suggestive of a common assembly pathway. In the case of peptides such as Aβ and IAPP, the non-fibrillar forms are essentially random coils that subsequently convert to a β-sheet under different conditions of pH and concentration.

CD provides a global view of the peptide secondary structure over an extended time course that can be used to monitor the amyloid conformational transition (i.e., diagnostic absorption minima for β-conformation at 218 nm). IAPP is initially treated with hexafluroisopropanol (HFIP) and trifluroacetic acid (TFA), to ensure it is in a monomeric form. IAPP 1-37 (50 μM) in the presence of inhibitors (50 μM-1.0 mM) are compared to controls, with spectra collected twice daily for a period of 72 hours. (β-sheet conversion and precipitation occurs at 48-72 hrs depending on conditions used.) Inhibitors can be evaluated for their ability to maintain IAPP in a random or non-β conformation. This can be determined by direct comparisons of the spectra obtained, and by quantitative analyses using deconvolution algorithms (e.g., Jasco J720 spectropolarimeter contained in the inventor's laboratory, and equipped with Spectra Manager).

2. Negative Stain Electron Microscopy: IAPP amyloid morphology and relative density of the fibrils can be examined by transmission EM techniques. This provides information of the morphology of the aggregates formed (e.g., amorphous, fibrillar, truncated/protofilament structures, and/or abnormal lateral aggregation profiles). In addition, scanning of multiple grids and samples can be performed by an experienced EM technician who will assess the relative amount of fibrils formed in the presence of each inhibitor. This supporting evidence can be used to rank the relative activities of the inhibitors. Aliquots are taken from the samples used for CD analysis, and applied to pioloform coated grids, blotted dry, and stained with phosphotungstic acid (pH 7.0).

3. IAPP Amyloid Toxicity: Amyloid fibril cytotoxicity may be due to an apoptotic mechanism and/or mechanical disruption of the plasma membrane. The ability of inhibitors to prevent toxicity in cells exposed to exogenous amyloid is another indication of activity that is assessed. A rat insulinoma line (RIN-1056A) having β-cell-like characteristics is exposed to 10 μM IAPP 1-37 with inhibitors added simultaneously to the medium at 1-20 fold excess. IAPP and inhibitors are not pre-mixed, but are added separately to the cell culture medium to maximize the similarities to conditions in vivo.

Cell viability is quantified using the AlamarBlue assay (Biosource International) that provides an easily detectable fluorescent output that does not interfere with the amyloid-cell interactions. Toxicity is assayed both over the short (sampling every 2 hr over an 8-10 hr period) and long term (every 12 hr for a period of 6-7days). Data from these investigations will indicate if the inhibitors are able to regulate this more physiological action of IAPP.

4. Inhibition of Biosynthetic IAPP Fibrils: Previous studies have demonstrated that islets cultured from IAPP transgenic mice stimulated with glucose will generate extracellular amyloid fibrils. This represents a close approximation to an in vivo setting. The inhibitors of the present invention are examined for their ability to prevent formation of this biosynthetic IAPP amyloid.

For example, islet cultures from wild-type mice expressing the non-amyloidogenic peptide and transgenic mice over-expressing human IAPP are isolated using established protocols (Tsujimura et al., Transplantation, 74:1687-691, 2002). Islets are then co-cultured with the most active inhibitors, as identified by the CD/EM/toxicity assays (initially at 20-fold excess to obtain maximal effect). Following treatment, cells can be fixed, embedded, and sectioned for immunohistochemistry. IAPP amyloid surrounding islets, and within intercellular spaces, is visualized using thioflavin S (ThS) which is a fluorescent dye that specifically detects amyloid deposits. IAPP fibrils in multiple sections can be quantified using image-analysis protocols, as described previously for the immunization treatments of Alzheimer amyloid transgenic mice.

### Example 1

### Minimal Inhibitory Peptide Sequence

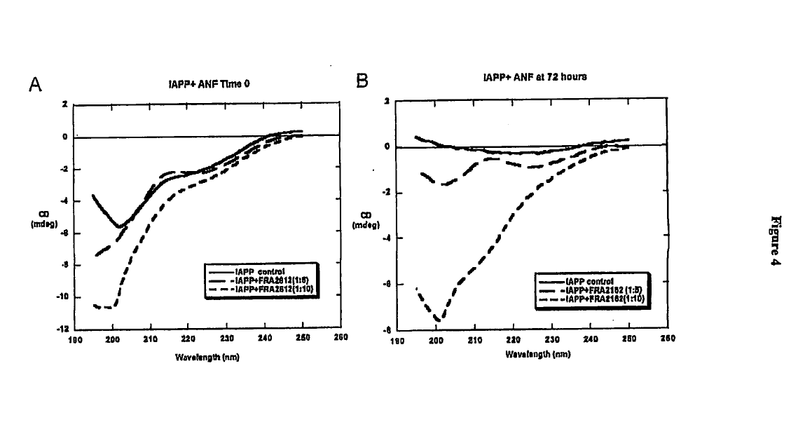

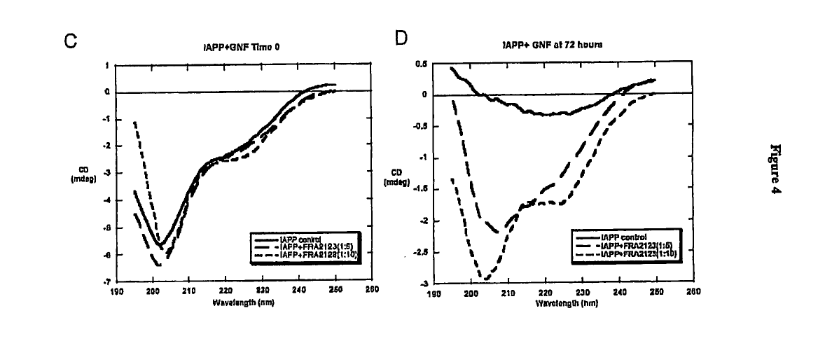

Hexapeptides are considered too large to be effective inhibitors under physiological conditions; a smaller active subunit contained within the active hexapeptide ANFLVH (SEQ. ID. NO 11) is more useful. To identify such smaller active subunits, systematic truncation of individual hexapeptides was performed. The complete series of truncated ANFLVH (13-18) (FIG. 3) was investigated using the in vitro assays as described above (CD, EM, and cell toxicity), over a typical dosing range (molar ratio from 1:1-1:20). The results from this study identified the more active domain(s) within the larger hexapeptide. Three sub-fragments—ANFLV (SEQ. ID. NO. 22), ANFL (SEQ. ID. NO. 23), and ANF (SEQ. ID. NO. 24)—were synthesized and examined in the RIN-cell toxicity assay using exogenous IAPP (FIG. 6). This initial study demonstrated that truncated peptides can exhibit activity comparable to the longer precursors. Each of these peptides showed activity, the tripeptide showing greater activity than the tetra- or penta-peptide. Accordingly, the inventor has demonstrated that truncated peptides, as opposed to the original hexapeptide ANFLVH, show anti-amyloid activity.

### Example 2

### Residue Specificity and Optimization

Not wishing to be bound by any particular theory of the peptide fragments' mechanism of action, it is proposed that binding to IAPP disrupts the normal packing of the polypeptide backbone and/or side-chain interactions within the fibrils. However, the exact structural basis for these interactions is not known, and it is conceivable that slight modifications in the inhibitory peptides may improve binding and/or render the peptides more effective at disrupting the amyloid polymer packing. To address this possibility, a combinatorial approach was examined by substitution of each residue within the active sequence. All naturally-occurring amino acids can be used to generate a peptide library in an effort to optimize the activity of the peptide inhibitors.

Based upon the results from the truncation study, the most active peptides (e.g., tripeptides, tetrapeptides) can be systematically substituted at each residue with a different amino acid. For example, with respect to the tripeptide ANF (residues 13-15), substitutions can be made as follows: ANX (SEQ. ID. NO. 28), AXN (SEQ. ID. NO. 29), and XNF (SEQ. ID. NO. 30), with X corresponding to Gly, Ala, Val, Leu, Ile, Ser, Thr, Met, Asp, Asn, Glu, Gin, Arg, Lys, His, Phe, Tyr, Trp, or Pro. This will generate 57 different derivatives that can be examined using an in vitro assay system, such as described below (CD, EM, and cell toxicity). Additionally, this will allow optimization of inhibitor activity, and also provide valuable structural information regarding their potential mechanism of action. In the present case, the tripeptide ANF was substituted with G at each of its amino acid positions, to generate the tripeptides GNF, AGF, and ANG.

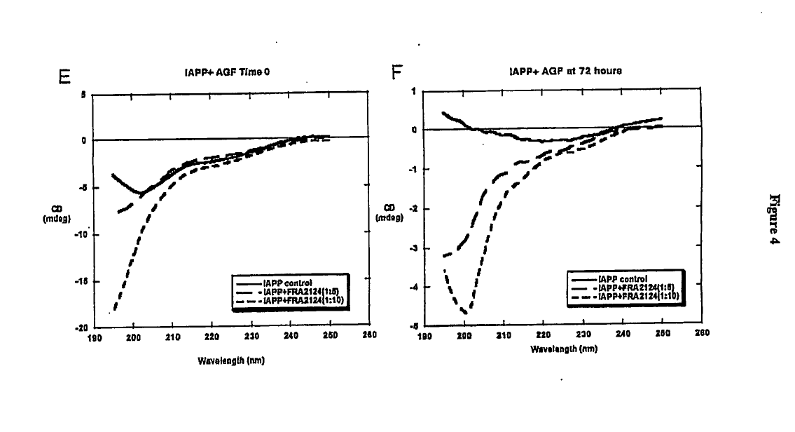

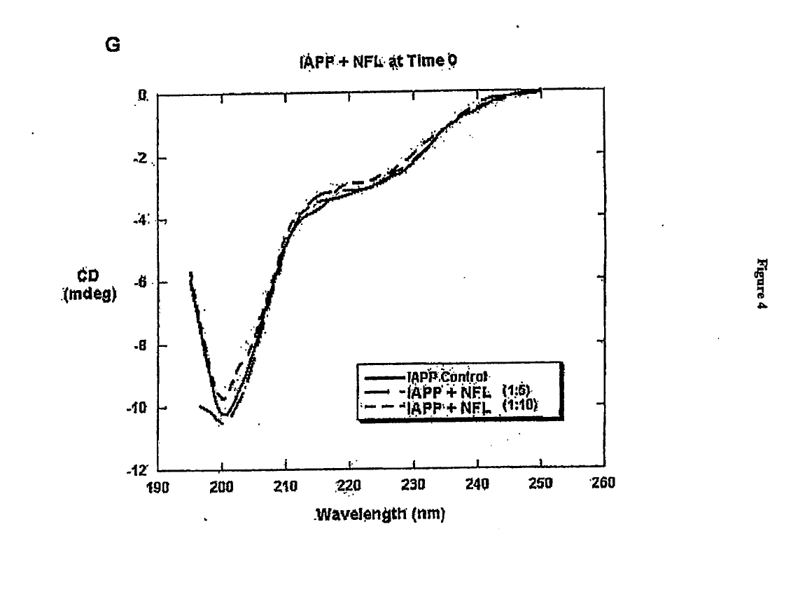

FIGS. 4A-H set forth circular dichroism (CD) data for the truncated tripeptide, ANF, indicating the transition from random coil to beta-sheet. FIGS. 4A and 4B show that ANF inhibited this conformational change when combined with IAPP at a molar ratio of 1:10. Modifications of this initial inhibitory sequence, to generate the peptides GNF (SEQ. ID. NO. 25) (FIGS. 4C and 4D) and AGF (SEQ. ID. NO. 26) (FIGS. 4E and 4F), resulted in similar, but more potent, activities at significantly lower molar ratios. Data for truncated peptide NFL (SEQ. ID. NO. 33) are shown in FIGS. 4G and 4H.

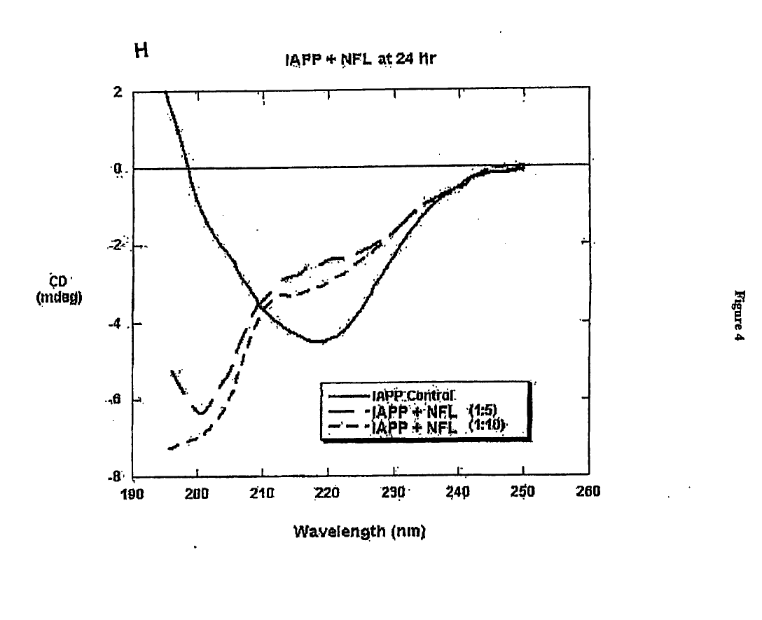

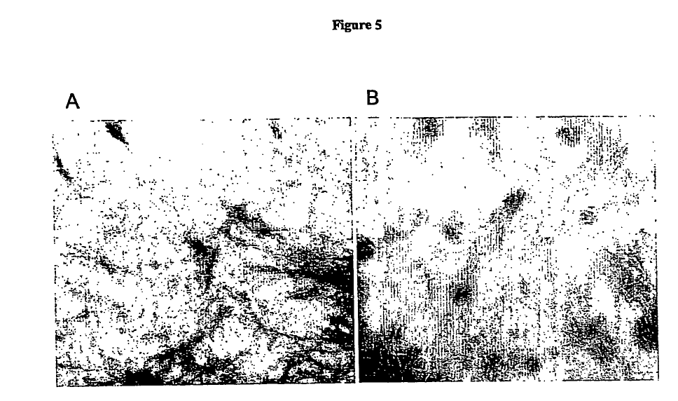

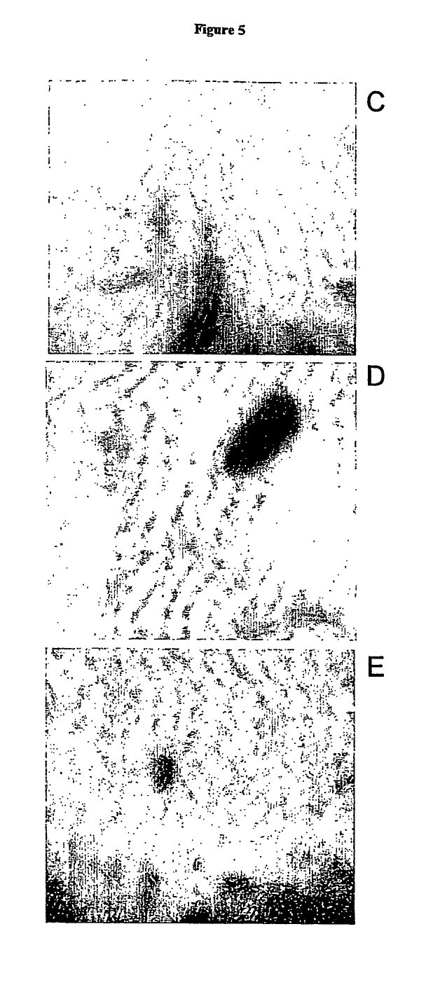

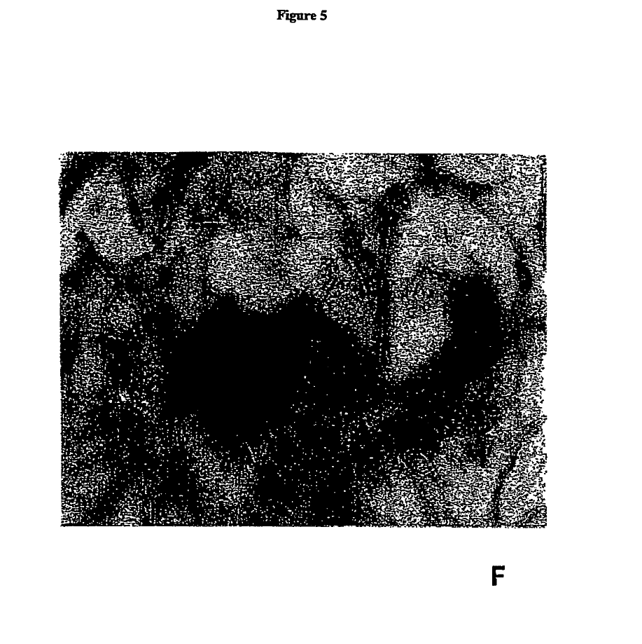

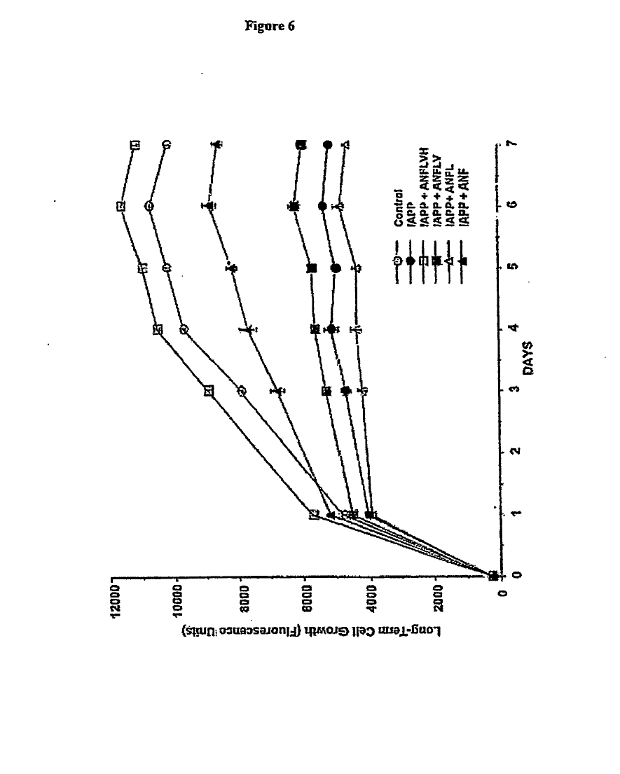

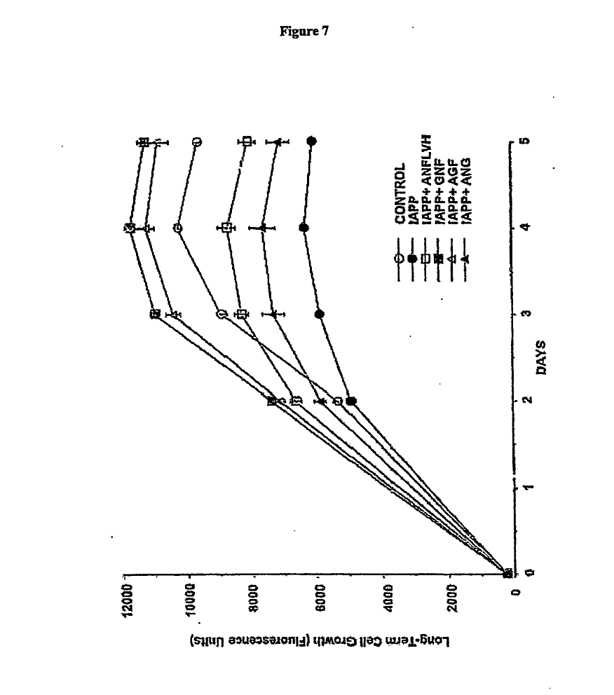

FIGS. 5A-F are electron micrographs from negatively-stained preparations of the full-length IAPP (FIG. 5A), showing the dense network of amyloid fibrils. FIG. 6B illustrates that the inhibitory peptide, ANF, reduced the relative levels of fibrils, and altered the morphology of the aggregates which were formed. Examination of the substituted peptides GNF (SEQ. ID. NO. 25) (FIG. 5C) and AGF (SEQ. ID. NO. 26) (FIG. 5D) indicated a significantly reduced density of fibrils, which is consistent with the CD data (FIG. 4). Results for active peptide NFL (SEQ. ID. NO. 33) are shown in FIG. 5F. Amorphous aggregates were observed in some instances, but virtually no recognizable amyloid-like fibrils were observed. The additional ANG peptide (SEQ. ID. NO. 27) (FIG. 5E) appeared to have a lower activity based upon the presence of multiple aggregate forms; this may explain the lower activity in the toxicity assay (FIG. 7).

FIGS. 6 and 7 illustrate the toxicity data results for the peptides tested. FIG. 7 illustrates the results for the optimized tripeptides. The results are consistent with what would be predictable from the CD and EM studies. GNF (SEQ. ID. NO. 25) and AGF (SEQ. ID. NO. 26) appeared to have more activity than ANG (SEQ. ID. NO. 27) or ANF (SEQ. ID. NO. 24).

### Example 3

### Small Molecule Analogues of Peptide Inhibitors

Due to low bioavailability and peripheral degradation, peptide treatments present a number of problems for drug development. Translating peptides into a small molecule mimetic is often difficult, due to the typical size of the active sequences (hexamers and larger). Tripeptides, however, display significant activity, and are in a molecular weight range more tractable in terms of predicting the active structure and equivalent small molecule analogues.

The approach to generating small molecule analogues involves molecular modeling and energy minimization, in order to obtain a likely structure of the peptide fragment. Using this as a template, it is possible to synthesize chemically a small organic molecule that resembles this structure. Any inhibitory molecule may then be optimized using standard structure-activity-relationship approaches based upon the original organic compounds.

Alternatively, a direct structural approach could be taken where, for example, the ANF peptide is combined with full-length IAPP. The ANF would, by virtue of its inhibitory properties, maintain IAPP in a soluble state that could be amenable to NMR analysis. This strategy could generate a 3-D molecular structure of the bound inhibitor, thereby revealing the active conformation. Information of this sort could then be used to design more effective mimetics of the ANF peptide.

### Example 4

### Examining the Effects of Amyloid Inhibitors on Islet Survival

Culturing human islets is often problematic and yields (e.g., from donors used for transplant purposes) are often highly variable. It has been proposed that this could be due to intrinsic pancreatic proteases, such as trypsin, and/or a sensitivity to oxidative stress. In contrast, mouse islets appear to be more robust, and are stable in culture for several days. Although post-mortem effects may come into play, one possible explanation is that the propensity of human IAPP to form amyloid may contribute to the observed cell death. The inability of murine IAPP to undergo this transformation, and its more soluble nature, may provide a protective effect leading to more viable cells. This is supported by the observation that amyloid was formed rapidly in transplanted islets expressing human IAPP, promoting cell death. Amyloid-related toxicity, therefore, may have implications for transplantation therapies which are currently being used to treat type 2 diabetes.

To test this hypothesis, islets were isolated from different transgenic animals, and their survivability in culture was examined. Transgenic mice were generated using standard methods and a full-length human IAPP (including the pro-sequence) with expression driven by the rat insulin promoter (FIG. 8). The transgenic mice also expressed mouse IAPP, which can influence the process of amyloidogenesis. To avoid complication arising from expression of mouse IAPP, the animals were also crossed onto an IAPP knockout line to obtain a humanized version of the transgene, at least as far as IAPP was concerned. The method used herein was similar to that described in Verchere et al., Proc. Natl. Acad. Sci. USA, 93:3492-496, 1996, with some modifications.

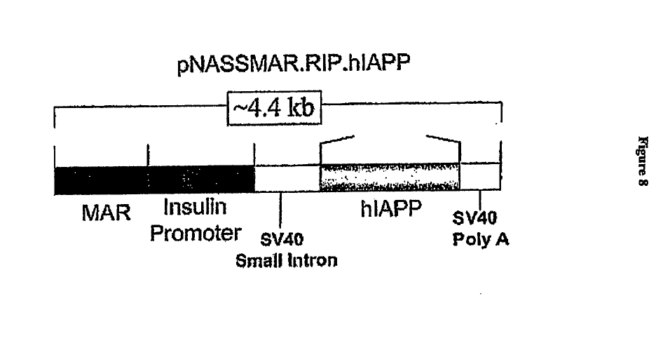

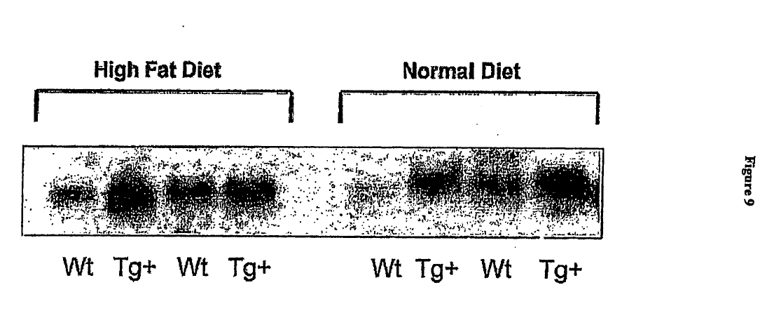

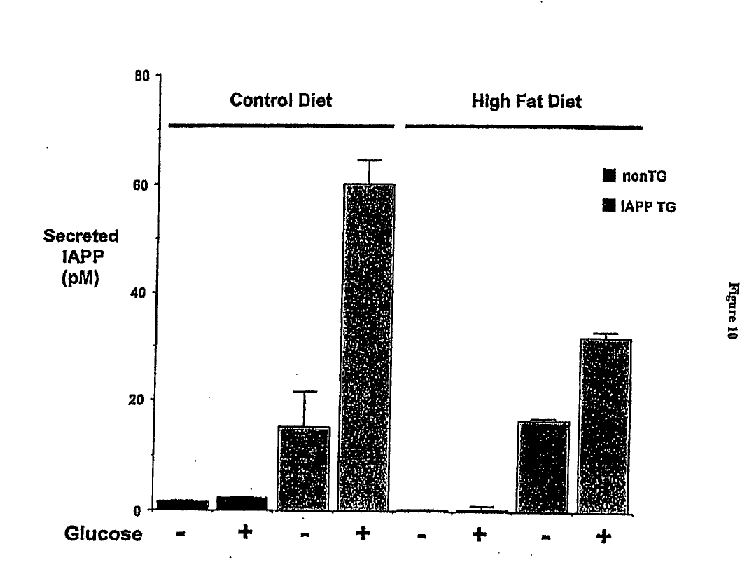

Initial studies using transgenic islets revealed a high level of IAPP secretion, as measured by a commercial ELISA (Linco Research) (FIG. 9). Interestingly, islets extracted from animals fed on a high-fat diet appeared to have low levels of secretions, possibly due to p cell exhaustion (FIG. 10). The diets used in these studies were similar to that described in Verchere et al., Proc. Natl. Acad. Sci. USA, 93:3492-496, 1996.

For the investigation, islets were extracted from: (1) wild-type control mice; (2) transgenics over-expressing human IAPP on a normal mouse background; and (3) transgenics on an IAPP knockout background. There should be a defined gradient in viability for these cultures, with the lowest survival being seen for islets on the IAPP-ablated background. This represents the closest approximation of a human culture, and should have the greatest amyloid load. Intermediate viability should be observed for islets over-expressing human IAPP, as attenuation of the amyloid pathway by the endogenous murine protein is anticipated.

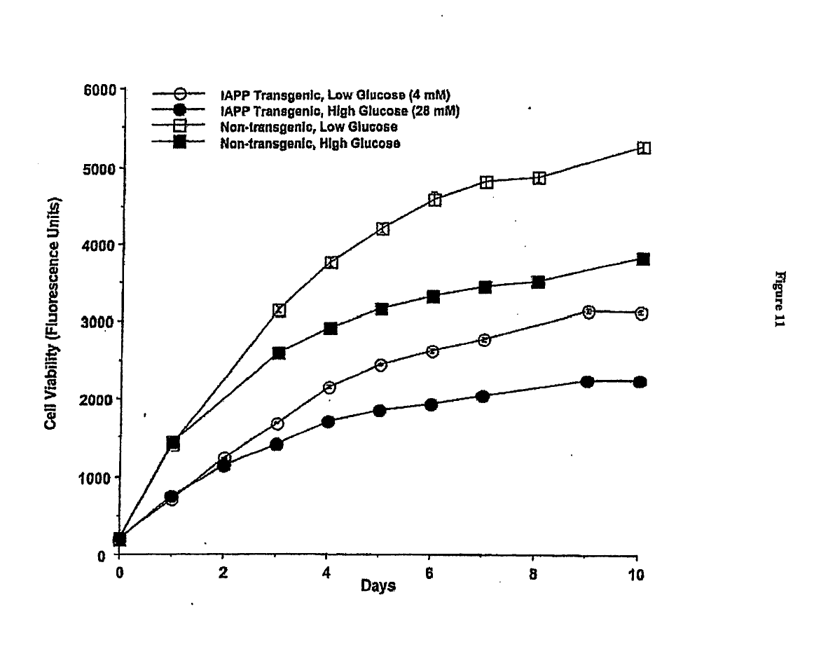

As another complicating factor, islets often exhibit necrosis within their core, most likely due to poor perfusion of these cells. To avoid this problem, viability analysis was performed on dissociated islets by trypsinization and subsequent passage of the cells through a 60 μm spectra mesh (Spectrum Labs Inc.). Dissociated islets were cultured in high (16.7 mmol/l) glucose (which is sufficient to stimulate IAPP fibril formation), and then compared to cells exposed to low (4.2 mmol/l) glucose. As with the toxicity assays, cell viability was determined over the course of 3-4 days using the AlamarBlue assay. Results of this study are presented in FIG. 11.

To confirm that cell death/survival correlates with amyloid load, fixed but unpermeabilized cells were stained for human IAPP using specific antibody, and examined by immunofluorescence. This permitted estimation of the amount of extracellular amyloid that was deposited in/around cells and in association with their plasma membranes. The polyclonal antibody used in the present invention was generated using a synthetic peptide antigen corresponding to residues 8-37 of the human IAPP. This was used to immunize rabbits, and antibodies were produced using a standard protocol.

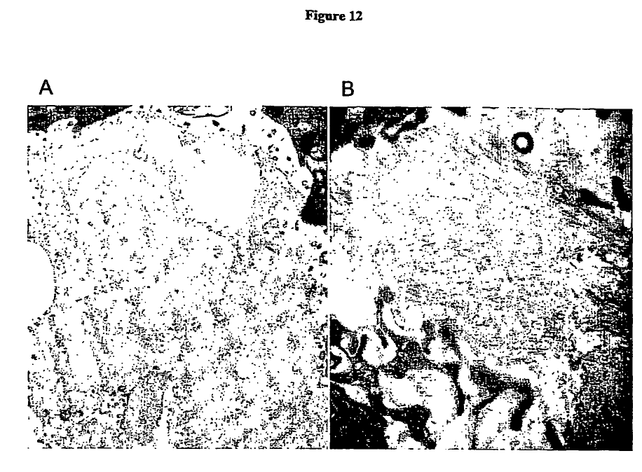

Additionally, electron microscopy images were taken of cultured islets isolated from transgenic mice expressing the human IAPP protein (FIG. 12). IAPP amyloid fibrils are visible in the interstitial spaces between the cells (center of the image) and radiating out of the plasma membranes. These are typical amyloid-like fibrils that have been observed in similar islet cultures derived from transgenic mice (de Koning et al., Proc. Natl. Acad. Sci. USA, 91:8467-471, 1994; de Koning, Diabetologia, 36:378-84, 1993).

This investigation can be extended, in order to examine if the most active IAPP inhibitors (derived from peptide studies and Innodia small molecules) are able to increase islet survival. The outcomes from the investigation as a whole can: (1) provide additional support for a significant role of IAPP amyloid in islet cell death; (2) further validate the effectiveness of the inhibitors which have been developed; and (3) provide a new and potentially important tool for the treatment of human islets that can be used to isolate cleaner, and more viable, preparations from donors for transplantation therapies currently being used for type 2 diabetes.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents, and patent applications are herein incorporated by reference in their entirety to the same extent as if each individual publication, patent, or patent application was specifically and individually indicated to be incorporated by reference in its entirety.

### INDEX TO SEQUENCE IDENTIFICATION NUMBERS

| | |
|---|---|
| SEQ. ID. NO. | DESCRIPTION |

| | |
|---|---|
| 1 | Human IAPP (37 amino acids (aa)) |
| 2 | Mouse IAPP - 37 aa |
| 3 | aa 8-18 of hIAPP |
| 4 | aa 20-29 of hIAPP |
| 5 | aa 31-37 of hIAPP |
| 6 | ATQRLA |
| 7 | TQRLAN |
| 8 | QRLANF |
| 9 | RLANFL |
| 10 | LANFLV |
| 11 | ANFLVH |
| 12 | NFLVHS |
| 13 | FLVHSS |
| 14 | SSNNFG |
| 15 | SNNFGA |
| 16 | NNFGAI |
| 17 | NEGAIL |
| 18 | FGAILS |
| 19 | GAILSS |
| 20 | AILSST |
| 21 | ILSSTN |
| 22 | ANFLV |
| 23 | ANFL |
| 24 | ANF |
| 25 | GNF |
| 26 | AGF |
| 27 | ANG |
| 28 | ANX, where X is any aa |
| 29 | AXF, where X is any aa |
| 30 | XNF, where X is any aa |
| 31 | NFLVH |
| 32 | FLVH |
| 33 | NFL |
| 34 | LVH |
| 35 | FLV |
| 36 | GSNKGAIIGL (β-IAPP, 25-34) |
| 37 | HVAAGAVVGG (PrP, 110-119) |
| 38 | ATQRLANFLVHSS |
| 39 | SSNNFGAILSSTN |

1. An antifibrillogenic agent for inhibiting amyloidosis and/or for cytoprotection, comprising a peptide selected from the group consisting of penta-, tetra-, and tri-peptides of truncated ANFLVH (SEQ. ID. NO. 11), or an isomer thereof, a retro or a retro-inverso isomer thereof, a peptidomimetic thereof, or a salt thereof.

2. The antifibrillogenic agent of claim 1, wherein said peptide is ANFLV (SEQ. ID. NO. 22), ANF (SEQ. ID. NO. 24), or NFL (SEQ. ID. NO. 33), an isomer thereof, a retro or a retro-inverso isomer thereof, a peptidomimetic thereof, or a salt thereof.

3. The antifibrillogenic agent of claim 1, wherein the agent comprises a tripeptide selected from the group consisting of ANF (SEQ. ID. NO. 24), ANX (SEQ. ID. NO. 28), AXF (SEQ. ID. NO. 29), and XNF (SEQ. ID. NO. 30), where X is any amino acid except cysteine, or an isomer thereof, a retro or a retro-inverso isomer thereof, a peptidomimetic thereof, or a salt thereof.

4. The antifibrillogenic agent of claim 3, wherein the tripeptide is selected from the group consisting of ANF (SEQ. ID. NO. 24), GNF (SEQ. ID. NO. 25), and AGF (SEQ. ID. NO. 26), or an isomer thereof, a retro or a retro-inverso isomer thereof, a peptidomimetic thereof, or a salt thereof.

5. (canceled)

6. (canceled)

7. (canceled)

8. (canceled)

9. The peptide of claim 1, wherein said peptide is ANFLV (SEQ. ID. NO. 22) or ANF (SEQ. ID. NO. 24).

10. The peptide of claim 1, wherein said peptide is a tripeptide selected from the group consisting of ANF (SEQ. ID. NO. 24), ANX (SEQ. ID. NO. 28), AXF (SEQ. ID. NO. 29), and XNF (SEQ. ID. NO. 30), where X is any amino acid except cysteine, or an isomer thereof, a retro or a retro-inverso isomer thereof, a peptidomimetic thereof, or a salt thereof.

11. The peptide of claim 10, wherein the peptide is selected from the group consisting of ANF (SEQ. ID. NO. 24), GNF (SEQ. ID. NO. 25), and AGF (SEQ. ID. NO. 26), or an isomer thereof, a retro or a retro-inverso isomer thereof, a peptidomimetic thereof, or a salt thereof.

12. The peptide of claim 11, wherein said sequence is ANF (SEQ. ID. NO. 24).

13. The tripeptide of claim 10, wherein said amyloidosis is IAPP-related.

14. The tripeptide of claim 10, wherein said amyloidosis is type 1 or type 2 diabetes.

15. A composition for inhibiting amyloidosis and/or for cytoprotection, comprising a therapeutically-effective amount of the peptide of claim 1 in association with a pharmaceutically-acceptable carrier.

16. A composition for inhibiting amyloidosis and/or for cytoprotection, comprising a therapeutically-effective amount of the peptide of any one of claim 10 in association with a pharmaceutically-acceptable carrier.

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. A method for the treatment of amyloidosis disorders in a patient, comprising administering to said patient a therapeutically-effective amount of the antifibrillogenic agent of claim 1.

33. The method of claim 32, wherein said amyloidosis disorder is IAPP-related.

34. The method of claim 33, wherein said amyloidosis disorder is type 1 or type 2 diabetes.

35. The method of claim 34, wherein said antifibrillogenic agent is administered in conjunction with another agent selected from the group consisting of insulin, sulfonylurea, and glucose sensitizers.

36. (canceled)

37. (canceled)

38. (canceled)

39. (canceled)

40. A process for the preparation of cells suitable for transplantation into a mammal, which cells are capable of forming amyloid deposits, said process comprising contacting cells in vitro with the antifibrillogenic agent of claim 1 for inhibiting amyloid deposit formation.

41. The process of claim 40, wherein said antifibrillogenic agent causes breakdown of amyloid deposits, the deposits having been formed by said cells prior to said contact.

42. The process of claim 40, wherein said cells are cultured in the presence of said antifibrillogenic agent.

43. The process of claim 40, wherein said amyloid deposits comprise IAPP amyloid.

44. The process of claim 40, wherein said amyloid deposits are associated with type 1 or type 2 diabetes.

45. The process of claim 40, wherein said cells, prior to treatment, form amyloid deposits.

46. Cells suitable for transplantation into a mammal, which have been prepared by the process of claim 40.

47. A method for treating a type 1 or type 2 diabetes patient after transplantation, said method comprising the step of administering in vivo to said patient the antifibrillogenic agent of claim 1 for inhibiting, preventing, and/or reducing amyloid deposit formation and amyloidosis.

48. The method of claim 47, wherein said amyloid deposit formation and/or amyloidosis is IAPP-related.

49. The method of claim 47, wherein said composition is administered in conjunction with another agent selected from the group consisting of insulin, sulfonylurea, and glucose sensitizers.

50. A method for inhibiting amyloidosis and/or for cytoprotection, comprising administering to a subject a therapeutically-effective amount of the antifibrillogenic agent of any one of claims 1 to 7, wherein said antifibrillogenic agent prevents or reduces amyloid deposition.

51. (canceled)

52. (canceled)

53. (canceled)

54. (canceled)

55. (canceled)

56. (canceled)

57. A method for identifying an optimized peptide for inhibition of amyloidosis, comprising the steps of:
(a) choosing an original peptide selected from the group consisting of ANF (SEQ. ID. NO. 24), GNF (SEQ. ID. NO. 25), AGF (SEQ. ID. NO. 26), and NFL (SEQ. ID. NO. 33),
(b) systematically substituting at each residue a different amino acid,
(c) testing the ability of each derivative to inhibit amyloid fibril formation, and
(d) comparing the inhibition of each derivative with the inhibition of the original peptide, wherein an increase in inhibition of the derivative as compared with the original peptide indicates an optimized peptide.

58. The method of claim 57, wherein the different amino acid is chosen from the group consisting of Gly, Ala, Val, Leu, Ile, Ser, Thr, Met, Asp, Asn, Glu, Gln, Arg, Lys, His, Phe, Tyr, Trp, and Pro.

59. The method of claim 57, wherein the original peptide is ANF (SEQ. ID. NO. 24).

60. The method of claim 57, wherein the testing for inhibition comprises at least one in vitro assay system selected from the group consisting of CD, EM, and cell toxicity.

61. The optimized peptide identified using the method of claim 57.
